(19) United States
(12) Patent Application Publication
(10) Pub. No.: US 2014/0335154 A1
(43) Pub. Date: Nov. 13, 2014

(54) METHODS AND FORMULATIONS TO ACHIEVE TUMOR TARGETED DOUBLE STRANDED RNA MEDIATED CELL DEATH

(71) Applicant: MULTICELL IMMUNOTHERAPEUTICS, INC., WOONSOCKET, RI (US)

(72) Inventor: Simona Bot, Beverly Hills, CA (US)

(73) Assignee: MULTICELL IMMUNOTHERAPEUTICS, INC., WOONSOCKET, RI (US)

(21) Appl. No.: 14/207,454

(22) Filed: Mar. 12, 2014

Related U.S. Application Data
(60) Provisional application No. 61/777,972, filed on Mar. 12, 2013.
(60) Provisional application No. 61/903,202, filed on Nov. 12, 2013.
(60) Provisional application No. 61/926,618, filed on Jan. 13, 2014.

(51) Int. Cl. A61K 31/713; C12N 15/113; A61K 45/06

(52) U.S. Cl. CPC A61K 31/713; A61K 45/06; C12N 15/113; C12N 2310/17; C12N 2310/51
USPC 424/450; 536/24.5; 514/44 R

(57) ABSTRACT

A composition having double stranded ribonucleic acid (dsRNA) molecules is provided. The composition induces tumor cell death or suppresses tumor growth. The double stranded RNA molecules contain equal to or less than 15 base pairs. Methods for delivery of the composition are also disclosed.

### PRIORITY CLAIM AND CROSS-REFERENCE TO RELATED APPLICATIONS

This application claims priority benefit of U.S. Provisional Patent Application No. 61/777,972 (Docket #74-160), entitled “METHODS AND FORMULATIONS TO ACHIEVE TUMOR TARGETED DOUBLE STRANDED RNA MEDIATED IMMUNOGENIC CELL DEATH,” filed on Mar. 12, 2013, by SIMONA BOT, which is incorporated herein by reference; this application also claims priority benefit of U.S. Provisional Patent Application No. 61/903,202 (Docket #74-161), entitled “METHODS AND COMPOSITIONS TO TREAT CANCER BY USING TARGETED NANOSTRUCTURES,” filed on Nov. 12, 2013, by SIMONA BOT, which is incorporated herein by reference; this application also claims priority benefit of U.S. Provisional Patent Application No. 61/926,618 (Docket #74-162), entitled “METHODS AND COMPOSITIONS TO INTERFERE WITH BASIC CELLULAR PROCESSES IN CANCEROUS CELLS USING VERY SMALL DSRNA (VSRNAS) MOLECULES,” filed on Jan. 13, 2014, by SIMONA BOT, and the contents of all of the above listed applications are incorporated herein by reference, in their entirety.

### FIELD

This specification generally relates to double stranded RNA.

### BACKGROUND

The subject matter discussed in the background section should not be assumed to be prior art merely as a result of its mention in the background section. Similarly, a problem and the understanding of the causes of a problem mentioned in the background section or associated with the subject matter of the background section should not be assumed to have been previously recognized in the prior art. The subject matter in the background section may merely represent different approaches, which in and of themselves may also be inventions.

Hepatocellular carcinoma (HCC) is the most common form of primary liver cancer and a leading cause of cancer death. Current pharmacological approaches for the treatment of human HCC are very limited in their efficacy and current pharmacological approaches do not provide durable control of disease.

A potential role for noncoding double stranded ribonucleic acids (dsRNAs) in the control of tumors has recently emerged in a variety of models that demonstrate the ability of noncoding double stranded RNAs to stimulate an innate immune response [1,2], or directly induce apoptosis [3]. Noncoding dsRNA stimulate immunity and are capable of inducing cell death in certain types of cells by engaging various signal transduction pathways through Toll-like Receptors (TLRs), melanoma differentiation associated gene 5 (MDA5) and retinoic acid inducible gene-I (RIG-I). Depending on the chemical structure and molecular weight, synthetic RNAs could differentially trigger signal transduction pathways and additional pathways yet to be characterized [1], providing an opportunity to discover, optimize and translate novel immune interventions for hepatocellular carcinoma and other unmet medical needs.

Earlier studies showed that unfractionated polyA:polyU spanning low and/or high molecular weight molecules could effectively license antigen presenting cells to cross-prime Tc1 responses and facilitate efficacious anti-tumor immunity [4,5]. This raises the possibility that immune stimulating dsRNAs are effective adjunctive therapy to any small molecule targeted therapy (such as tyrosine kinase inhibitors—TKIs) that results in release of endogenous tumor antigen while interfering minimally with the immune competence [6,7].

No information is available regarding the in vitro or in vivo activity in liver cancer models. In addition, the molecular mechanism of action, or the receptors mediating the cellular effect of low molecular weight dsRNA have not been elucidated yet.

During the last few years, there has been significant progress in manufacturing of RNAs resulting in more cost effective and feasible manufacturing of molecules of exact size, instead of imprecise methods involving size fractionation of heterogenic pools that have been tested earlier in clinic [10, 11]. In addition, as an alternative to synthesis of dsRNA with a native chemical structure, there is now the possibility to synthesize analogues with increased in vitro and in vivo stability and substantially decreased manufacturing costs, respectively.

### BRIEF DESCRIPTION OF THE FIGURES

In the following drawings like reference numbers are used to refer to like elements. Although the following figures depict various examples of the embodiments of the invention, the invention is not limited to the examples depicted in the figures.

FIG. 1 shows mechanisms of recognition and action of dsRNAs with cytotoxic and immune modulating properties;

FIG. 2A shows the chemical structure of 5 base pairs polyadenylic-polyuridylic acid (polyA:polyU or pA:pU);

FIG. 2B shows the chemical structure of 2O′-methyl analogue of 5 base pairs polyA:polyU;

FIG. 3A shows enhanced anti-tumor cell and pro-inflammatory effects of low molecular weight dsRNA (<15 bps) on transformed monocytic human cells of bone marrow origin (THP-1 cells);

FIG. 3B shows that low molecular weight dsRNA (5 bps pA:pU) induce TNFalpha and IL-6 in human HCC cell line PLC/PRC/5 and transformed monocytic cells of bone marrow origin (THP-1 cells);

FIG. 4A shows that polyA:polyU of 5 bps has cell growth inhibition and death inducing properties in human HCC lines PLC/PRF/5, Huh7 and HepG2 in a dose-effect fashion, while 2O′-methyl polyA:polyU analogues of 5 bps shows an attenuated cytotstatic/cytotoxic profile;

FIG. 4B shows that polyA:polyU of 5 bps has cell growth inhibition and death inducing properties in primary human liver cancer cells P7NSG59410 and P31NSG55368, and mouse liver cancerous cell line in a dose-effect fashion, while 2O′-methyl polyA:polyU analogues of 5 bps shows an attenuated cytotstatic/cytotoxic profile;

FIG. 4C shows that polyA:polyU of 5 bps has cell growth inhibition and death inducing properties in THP-1 cells in a dose-effect fashion, while THLE2 cells emulating normal human liver cells were more refractory to 5 bp polyA:polyU; in addition, human primary fibroblasts were sensitive to 5 bp polyA:polyU;

FIG. 5A shows that polyA:polyU of 5 bps induces death of PLC/PRF/5 and Huh7 cells;

FIG. 5B shows that polyA:polyU of 5 bps induces cell death of HepG2 and THP-1 cells;

FIG. 6 shows that Lipofectamine formulated pA:pU for intracellular delivery is more biologically active than unformulated pA:pU in human liver cancer cell lines Huh7 and HepG2;

FIG. 7 shows the structure of one example of the formulated dsRNAs using biodegradable matrix;

FIG. 8 shows the structure of one example of the formulated dsRNAs using dendrimers; and

FIG. 9 shows the structure of another example of the formulated dsRNAs in liposomes.

### DETAILED DESCRIPTION

Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

### INCORPORATION BY REFERENCE

The references below are incorporated by reference in their entirety and may contain further details of some of the mechanism and compositions discussed in the rest of the specification:

dsRNA Related
1. Jin B, Sun T, Yu X H, Liu C Q, Yang Y X, Lu P, Fu S F, Qiu H B, Yeo A E. Immunomodulatory effects of dsRNA and its potential as vaccine adjuvant. J Biomed Biotechnol. 2010; 2010:690438;
2. Kleinman M E, Yamada K, Takeda A, Chandrasekaran V, Nozaki M, Baffi J Z, Albuquerque R J, Yamasaki S, Itaya M, Pan Y, Appukuttan B, Gibbs D, Yang Z, Karikó K, Ambati B K, Wilgus T A, DiPietro L A, Sakurai E, Zhang K, Smith J R, Taylor E W, Ambati J. Sequence- and target-independent angiogenesis suppression by siRNA via TLR3. Nature. 2008 Apr. 3; 452(7187):591-7;
3. Salaun B, Coste I, Rissoan M C, Lebecque S J, Renno T. TLR3 can directly trigger apoptosis in human cancer cells. J Immunol. 2006 Apr. 15; 176(8):4894-901;
4. Wang L, Smith D, Bot, S, Dellamary L, Bloom A, Bot A; Noncoding RNA danger motifs bridge innate and adaptive immunity and are potent adjuvants for vaccination. J Clin Invest. 110:1175-1184 (2002);
5. Bot A, Smith D, Phillips B, Bot S, Bona C, Zabhouani H. Immunologic control of tumors by in vivo Fcγ receptor-targeted antigen loading in conjunction with double-stranded RNA-mediated immune modulation. J Immunol. 176: 1363-1374 (2006);
6. van der Most R G, Currie A, Robinson B W, Lake R A. Cranking the immunologic engine with chemotherapy: using context to drive tumor antigen cross-presentation towards useful antitumor immunity. Cancer Res. 2006 Jan. 15; 66(2):601-4;
7. Ozao-Choy J, Ma G, Kao J, Wang G X, Meseck M, Sung M, Schwartz M, Divino C M, Pan P Y, Chen S H. The novel role of tyrosine kinase inhibitor in the reversal of immune suppression and modulation of tumor microenvironment for immune-based cancer therapies. Cancer Res. 2009 Mar. 15; 69(6):2514-22;
8. Mizuno K, Toyoda Y, Fukami T, Nakajima M, Yokoi T. Stimulation of pro-inflammatory responses by mebendazole in human monocytic THP-1 cells through an ERK signaling pathway. Arch Toxicol. 2010 Sep. 17;
9. Akazawa Y, Mott J L, Bronk S F, Werneburg N W, Kahraman A, Guicciardi M E, Meng X W, Kohno S, Shah V H, Kaufmann S H, McNiven M A, Gores G J. Death receptor 5 internalization is required for lysosomal permeabilization by TRAIL in malignant liver cell lines. Gastroenterology. 2009 June; 136(7):2365-2376.e1-7;
10. Lacour J, et al. Adjuvant treatment with polyadenylic-polyuridylic acid in operable breast cancer: updated results of a randomised trial. Br Med J (Clin Res Ed). 1984 Feb. 25; 288(6417):589-92;
11. Witt P L et al. Phase I/IB study of polyadenylic-polyuridylic acid in patients with advanced malignancies: clinical and biologic effects. J Interferon Cytokine Res. 1996 August; 16(8):631-5;
12. Simona Bot, Feng He, W. Gerald Newmin, Anand Ghanekar. Sharply discordant biological properties of synthetic noncoding dsRNA of different size: translational opportunities in cancer. CIMT Annual Meeting. May 23-25, 2012, Mainz, Germany;
13. Simona Bot, Feng He, W. Gerald Newmin, Anand Ghanekar. Short synthetic double stranded RNA with dual activity—oncolytic and immune modulatory—for hepatocellular carcinoma Abstract, American Society of Cellular Biology, Annual Conference, San Francisco, Dec. 15-19, 2012;

### Target Molecule Related

14. Khong F. Yoong, et al. Vascular Adhesion Protein-1 and ICAM-1 Support the Adhesion of Tumor-Infiltrating Lymphocytes to Tumor Endothelium in Human Hepatocellular Carcinoma. The Journal of Immunology, 1998, 160: 3978-3988;
15. Andrew X. Zhu, et al. First-in-Man Phase I Study of GC33, a Novel Recombinant Humanized Antibody Against Glypican-3, in Patients with Advanced Hepatocellular Carcinoma. Clin Cancer Res. 2013; 19:920-928;
16. Daniels T R, Delgado T, Rodriguez J A, Helguera G, Penichet M L. The transferrin receptor part I: Biology and targeting with cytotoxic antibodies for the treatment of cancer. Clin Immunol. 2006 November; 121(2):144-58;
17. Daniels T R, Delgado T, Helguera G, Penichet M L. The transferrin receptor part II: targeted delivery of therapeutic agents into cancer cells. Clin Immunol. 2006 November; 121(2):159-76;
18. Kraman M et al. Suppression of antitumor immunity by stromal cells expressing fibroblast activation protein-alpha. Science. 2010 Nov. 5; 330(6005):827-30;

### Formulation Related

19. Zhou et al. (2011) PAMAM dendrimer as potential delivery system for combined chemotherapeutic and microRNA-21 gene therapy. Non-Viral Gene Therapy. 21: 499-514;
20. Vladimir R. Muzykantov et al. Dynamic Factors Controlling Targeting Nanocarriers to Vascular Endothelium. Curr Drug Metab. 2012 January 1; 13(1): 70-81;
21. Ghosh R, Singh L C, Shohet J M, Gunaratne P H. A gold nanoparticle platform for the delivery of functional microRNAs into cancer cells. Biomaterials. 2013 January; 34(3):807-16;
22. Cubillos-Ruiz J R, Sempere L F, Conejo-Garcia J R. Good things come in small packages: Therapeutic anti-tumor immunity induced by microRNA nanoparticles. Oncoimmunology. 2012 Sep. 1; 1(6):968-970;
23. Piao L, Zhang M, Datta J, Xie X, Su T, Li H, Teknos T N, Pan Q. Lipid-based nanoparticle delivery of Pre-miR-107 inhibits the tumorigenicity of head and neck squamous cell carcinoma. Mol Ther. 2012 June; 20(6):1261-9;
24. Cheng C J, Saltzman W M. Polymer nanoparticle-mediated delivery of microRNA inhibition and alternative splicing. Mol Pharm. 2012 May 7; 9(5):1481-8;
25. Dong H, Lei J, Ju H, Zhi F, Wang H, Guo W, Zhu Z, Yan F. Target-cell-specific delivery, imaging, and detection of intracellular microRNA with a multifunctional SnO2 nanoprobe. Angew Chem Int Ed Engl. 2012 May 7; 51(19):4607-12;
26. Liu J, Wang B, Hartono S B, Liu T, Kantharidis P, Middelberg A P, Lu G Q, He L, Qiao S Z. Magnetic silica spheres with large nanopores for nucleic acid adsorption and cellular uptake. Biomaterials. 2012 January; 33(3):970-8;
27. Kim J K, Choi K J, Lee M, Jo M H, Kim S. Molecular imaging of a cancer-targeting theragnostics probe using a nucleolin aptamer- and microRNA-221 molecular beacon-conjugated nanoparticle. Biomaterials. 2012 January; 33(1):207-17;
28. Liang G F, Zhu Y L, Sun B, Hu F H, Tian T, Li S C, Xiao Z D. PLGA-based gene delivering nanoparticle enhance suppression effect of miRNA in HePG2 cells. Nanoscale Res Lett. 2011 Jul. 12; 6:447;
29. Chen Y, Zhu X, Zhang X, Liu B, Huang L. Nanoparticles modified with tumor-targeting scFv deliver siRNA and miRNA for cancer therapy. Mol Ther. 2010 September; 18(9):1650-6;
30. Ren Y, Kang C S, Yuan X B, Zhou X, Xu P, Han L, Wang G X, Jia Z, Zhong Y, Yu S, Sheng J, Pu P Y. Co-delivery of as-miR-21 and 5-FU by poly(amidoamine) dendrimer attenuates human glioma cell growth in vitro. J Biomater Sci Polym Ed. 2010; 21(3):303-14;
31. Jeanette Kaiser and Irene Kramer. Loading profile of topotecan into polyvinyl alcohol microspheres (DC Beadä) over a 7-day period. J Oncol Pharm Pract. 2012 18: 222 originally;
32. Chen L et al. Codelivery of zoledronic acid and double-stranded RNA from core-shell nanoparticles. International Journal of Nanomedicine. 2013:8 137-145;

### Oncolytic Viruses

33. Mahoney D J, Stojdl D F Molecular pathways: multimodal cancer-killing mechanisms employed by oncolytic vesiculoviruses. Clin Cancer Res. 2013 Feb. 15; 19(4):758-63;

### Biotechniques

34. Molecular Cloning A Laboratory Manual, 2nd Ed., ed. by Sambrook, Fritsch and Maniatis (Cold Spring Harbor Laboratory Press: 1989);
35. DNA Cloning, Volumes I and II (D. N. Glover ed., 1985);
36. Oligonucleotide Synthesis (M. J. Gait ed., 1984);
37. Mullis et al. U.S. Pat. No. 4,683,195;
38. Nucleic Acid Hybridization (B. D. Hames & S. J. Higgins eds. 1984);
39. Transcription And Translation (B. D. Hames & S. J. Higgins eds. 1984);
40. Culture Of Animal Cells (R. I. Freshney, Alan R. Liss, Inc., 1987);
41. Immobilized Cells And Enzymes (IRL Press, 1986); B. Perbal, A Practical Guide To Molecular Cloning (1984);
42. Methods In Enzymology (Academic Press, Inc., N.Y.);
43. Gene Transfer Vectors For Mammalian Cells (J. H. Miller and M. P. Calos eds., 1987, Cold Spring Harbor Laboratory);
44. Methods In Enzymology, Vols. 154 and 155 (Wu et al. eds.);
45. Immunochemical Methods In Cell And Molecular Biology (Mayer and Walker, eds., Academic Press, London, 1987);
46. Handbook Of Experimental Immunology, Volumes I-IV (D. M. Weir and C. C. Blackwell, eds., 1986);
47. Manipulating the Mouse Embryo, (Cold Spring Harbor Laboratory Press, Cold Spring Harbor, N.Y., 1986);

### Patents:

48. U.S. Pat. No. 8,372,818, Shir, et al. Feb. 12, 2013, Targeted double stranded RNA mediated cell killing;
49. U.S. Pat. No. 8,361,510. Lyon, et al. Jan. 29, 2013. Nanogels for cellular delivery of therapeutics;
50. U.S. Pat. No. 8,318,200 Sung, et al. Nov. 27, 2012. Pharmaceutical composition of nanoparticles;
51. U.S. Pat. No. 8,293,209. Segev Oct. 23, 2012. System for delivering therapeutic agents into living cells and cells nuclei;
52. U.S. Pat. No. 8,283,333 Yaworski, et al. Oct. 9, 2012. Lipid formulations for nucleic acid delivery;
53. United States Patent Application 20130028941. Altreuter; David H.; et al. Jan. 31, 2013. Synthetic Nanocarriers that Generate Humoral and Cytotoxic T Lymphocyte (Ctl) Immune Responses;
54. United States Patent Application 20130017223. Hope; Michael J; et al. Jan. 17, 2013. Methods and Compositions for Delivery of Nucleic Acids;
55. United States Patent Application 20120245076. Nakayama; Tomoko; et al. Sep. 27, 2012. Compositions and Methods for Delivering Rnai using Apoe;
56. United States Patent Application 20130011333. Yuan; Zhi-Min; et al. Jan. 10, 2013. Methods and Compositions for Nanoparticle-Mediated Cancer Cell-Targeted Delivery;
57. United States Patent Application 20120202871. Heyes et al. Aug. 9, 2012. Cationic Lipids and Methods for the Delivery of Therapeutic Agents;
58. United States Patent Application 2012/0122800. James Kadushin et al. Filing date: Aug. 10, 2009. Long-Acting DNA Dendrimers and Methods Thereof;
59. United States Patent Application 2011/0206611. Filed on Feb. 23, 2011. DNA Dendrimers as Thermal Ablation Devices;
60. United States Patent Application 2006/0160098. Filed on Dec. 9, 2004. Polymeric label molecules;

### Miscellaneous

61. Malgorzata Ferenc, Elzbieta Pedziwiatr-Werbicka, Katarzyna E. Nowak, Barbara Klajnert, Jean-Pierre Majoral and Maria Bryszewska. Phosphorus Dendrimers as Carriers of siRNA—Characterisation of Dendriplexes. Molecules 2013, 18(4), 4451-4466;
62. Swati Biswas and Vladimir P. Torchilin. Dendrimers for siRNA Delivery. Pharmaceuticals 2013, 6, 161-183;
63. Jiangyu Wu, Weizhe Huang, and Ziying He. Dendrimers as Carriers for siRNA Delivery and Gene Silencing: A Review. Hindawi Publishing Corporation: The Scientific World Journal Volume 2013, Article ID 630654;
64. Enrique Barrajón-Catalán, María P. Menéndez-Gutiérrez, Alberto Falco, Miguel Saceda,
Angela Catania and Vicente Micol (2011). Immunoliposomes: A Multipurpose Strategy in Breast Cancer Targeted Therapy, Breast Cancer—Current and Alternative Therapeutic Modalities, Prof. Esra Gunduz (Ed.), ISBN: 978-953-307-776-5, InTech;
65. T W Nilsen, J Grayzel, W Prensky. Dendritic nucleic acid structures. Journal of Theoretical Biology (Impact Factor: 2.35). August 1997; 187(2):273-84. DOI:10.1006/jtbi.1997.0446;
66. Daniel H. Kim and John J. Rossi. RNAi mechanisms and applications. BioTechniques 44:613-616 (April 2008) doi 10.2144/000112792;
67. Meister G, Tuschl T (2004). Mechanisms of gene silencing by double-stranded RNA. Nature 431: 343-349. PMID: 15372041;
68. CHIA-YING CHU and TARIQ M. RANA. Potent RNAi by short RNA triggers. RNA (2008), 14:1714-1719;
69. Dong Y, Love K T, Dorkin J R, Sirirungruang S, Zhang Y, Chen D, Bogorad R L, Yin H, Chen Y, Vegas A J, Alabi C A, Sahay G, Olejnik K T, Wang W, Schroeder A, Lytton-Jean A K, Siegwart D J, Akinc A, Barnes C, Barros S A, Carioto M, Fitzgerald K, Hettinger J, Kumar V, Novobrantseva T I, Qin J, Querbes W, Koteliansky V, Langer R, Anderson D G. Lipopeptide nanoparticles for potent and selective siRNA delivery in rodents and nonhuman primates. Proc Natl Acad Sci USA. 2014 Feb. 10;
70. Sandra D. Laufer, Anke Detzer, Georg Sczakiel, and Tobias Restle. Selected Strategies for the Delivery of siRNA In Vitro and In Vivo. V. A. Erdmann and J. Barciszewski (eds.), RNA Technologies and Their Applications, RNA Technologies, DOI 10.1007/978-3-642-12168-5_{—}2. Springer-Verlag Berlin Heidelberg 2010.

The specification recognizes the methods and formulations to achieve tumor targeted double stranded RNA mediated cell death. In brief, the embodiments of the present invention describe methods and compositions to achieve tumor targeted, double stranded RNA-mediated immunogenic cell death. A need addressed by at least some embodiments of the invention is directing the powerful biological effect of low molecular weight dsRNAs towards the tumor and away from normal tissues.

As used in the specification and claims, the singular form “a”, “an” and “the” are generic to plural references unless the context clearly dictates otherwise.

The term “double-stranded RNA” or “dsRNA” refers to two strands of ribonucleic acid comprised of the bases adenine, cytosine, uracil, guanine and inosine. The “dsRNA” may be entirely complimentary, partially complementary or a mixed nucleotide strand. More specifically, the duplex may encompass partially or totally annealed RNA strands, hairpin structures, completely matched or partially matched duplexes that encompass a combination of dsRNA and single stranded RNA portions.

As used herein, “low molecular weight dsRNA” means RNA strands composed of equal to or less than 15 base pairs. Although 5 base pairs are used as an example in many places in the specification, in one embodiment, “low molecular weight dsRNA” ranges between 1 to 14 base pairs. In another embodiment, “low molecular weight dsRNA” ranges between 2 to 10 base pairs. In another embodiment, “low molecular weight dsRNA” ranges between 10 and 15 base pairs. In another embodiment, “low molecular weight dsRNA” ranges between 2 to 5 base pairs. In another embodiment, “low molecular weight dsRNA” ranges between 5 and 10 base pairs. In yet another embodiment, “low molecular weight dsRNA” is 5 base pairs.

The term “pA:pU” refers to double stranded RNA where the RNA strand or segment is comprised of adenine (A) and uracil (U). In one embodiment, the RNA strand or segment is complementary. In other embodiments, the RNA strands or segments are not uniformly complementary.

The term “payload” refers to the main functional materials of the formulated particles, vehicles, or spheres, while “matrix” refers to the materials that form or support the structure or facilitate the delivery of the “payload.” In one embodiment, the “payload” is dsRNAs or analogues of dsRNA. In one embodiment, the “payload” may be covalently or non-covalently linked to the particle matrix. Alternatively, the “payload” may be encapsulated inside the particles or vehicles. In some embodiments, the payload itself may be assembled as a matrix that upon cellular internalization, liberates the dsRNA in a biologically active form.

As used herein, “analogue” refers to a chemical compound with a slightly altered chemical structure or composition, or with modifications. In one embodiment, “2O′-methyl analogue” is dsRNAs that has been modified to have a 2O′ methylation of the nucleic bases. In another embodiment, a “polyA:polyU analogue” is double stranded 2O′-methyl polyA:polyU.

As used herein, “Effective Dose (ED)50” refers to the “median effective dose”, which is the dose that produces a quantal effect (all or nothing) in 50% of the population that takes it (median referring to the 50% population base). The ED50 is commonly used as a measure of the reasonable expectancy of a drug effect, but does not necessarily represent the dose that a clinician might use. This depends on the need for the effect, and also the toxicity.

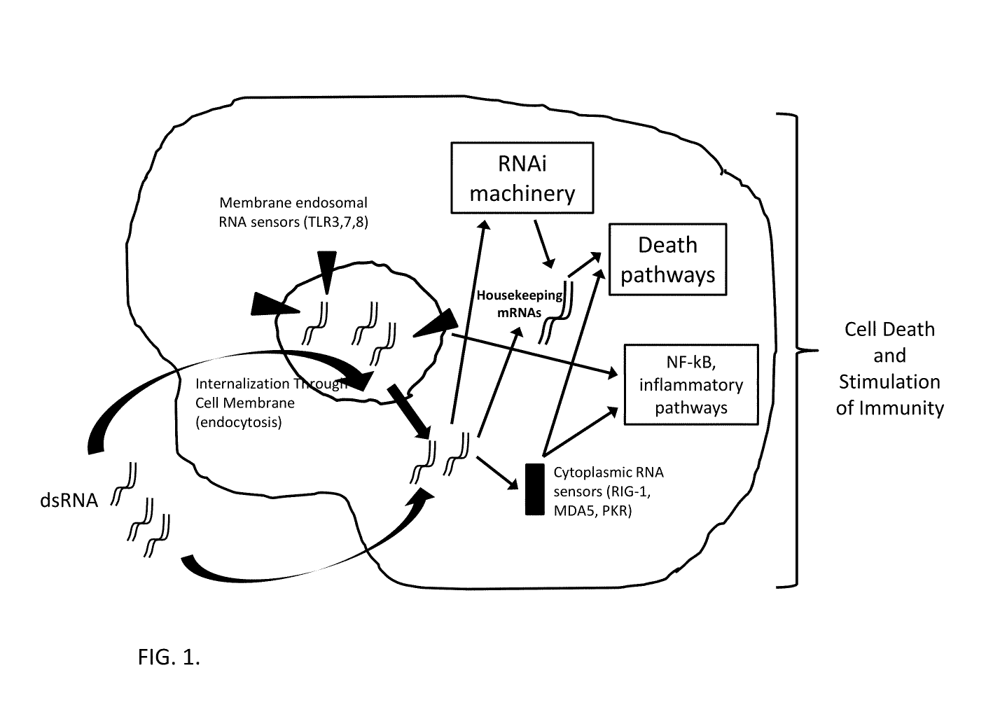

FIG. 1 shows pleiotropic mechanism of action of dsRNA with dual cytotoxic and immune enhancing properties. FIG. 1 is for illustration purpose only, and one skilled in the art would appreciate that FIG. 1 may not have all of the components or pathways for the mechanisms of dsRNA functionality, or may have other components or pathways instead of and/or in addition to those shown in FIG. 1. Double stranded RNAs could be internalized through cell membrane via endocytosis. Alternatively, dsRNAs with low molecular weight could enter the cell directly without utilizing a cell receptor. Double stranded RNAs could be recognized by cells of the mammalian immune system through extracellular receptors (membrane endosomal RNA sensors) that include TLR3, TLR7 and TLR8. The ligand-binding domains of the extracellular receptors face the endosomal compartment recognizing the dsRNAs before the dsRNAs enter the cytoplasm. The recognition of the dsRNA requires time-dependent endosomal maturation to trigger downstream signaling, which activates downstream inflammatory pathways, such as nuclear factor kappa-light-chain-enhancer of activated B cells (NF-kappaB) pathways. Most mammalian cells possess intracellular pathways that recognize dsRNA through cytoplasmic RNA sensors, such as Protein kinase RNA (PKR), MDA5 and RIG-I. Intracellular pathways that recognize dsRNA through cytoplasmic RNA sensors pathways can also recognize dsRNAs and activate inflammatory pathways, such as NF-kappaB pathways, as well as cell death pathways. The dsRNAs in the cytoplasm may bind to other messenger RNA (mRNA) molecules and either increase or decrease the activity the other mRNA. The cytoplasmic dsRNAs may also enter the RNA interference (RNAi) pathway, and the RNAi pathway causes the destruction of the mRNA molecules including housekeeping mRNAs which, upon destruction, activate cell death pathways.

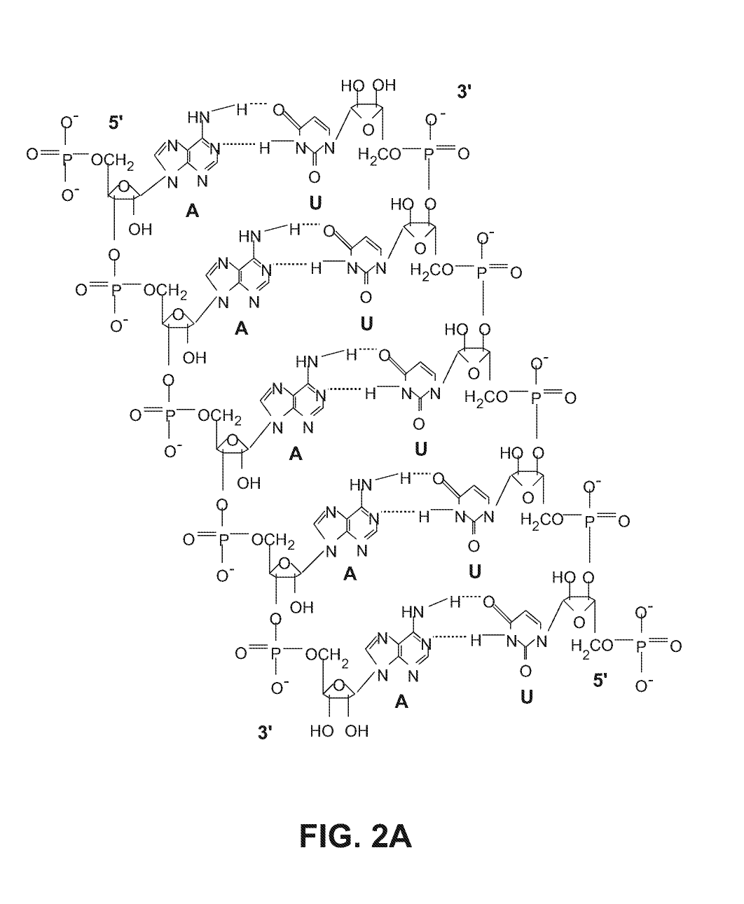

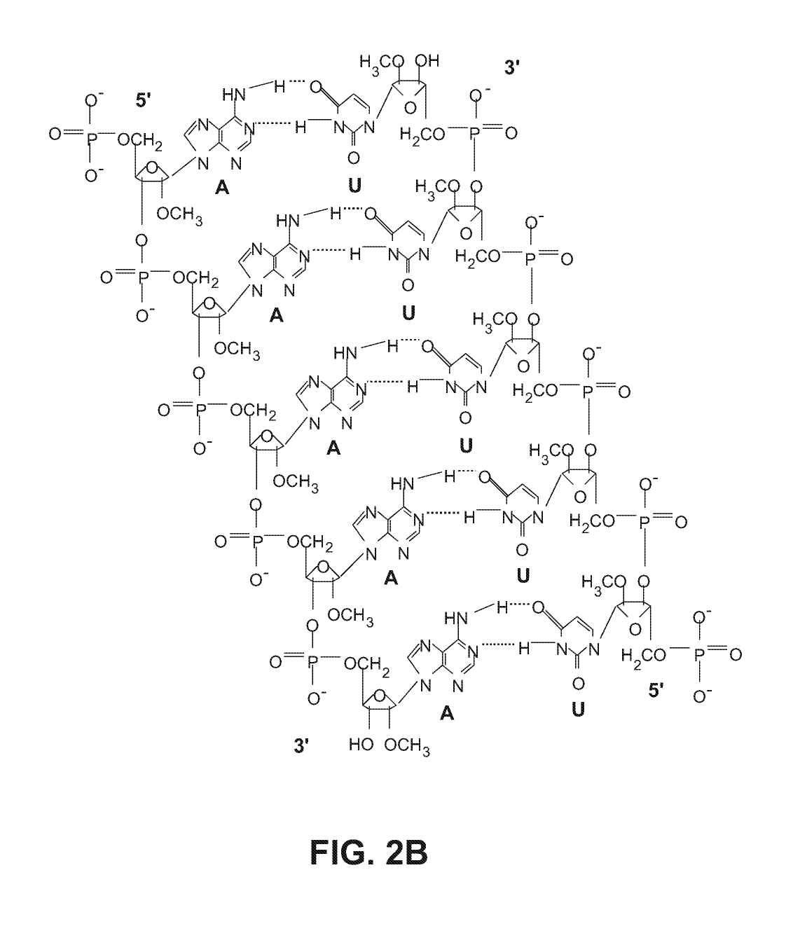

FIG. 2A shows the chemical structure of 5 base pairs polyA:polyU. In one embodiment, 5 base pairs of polyA:polyU contains two complementary strands of ribonucleotides, one strand of which contains five Adenosine monophosphates (also know as 5′-adenylic acid) linked via phosphodiester bonds, while the other strand of which contains five Uridine monophosphate (also known as 5′-uridylic acid) linked via phosphodiester bonds. The double stranded polyA:polyU have base pairs A:U linked by hydrogen bounds, acting as the building blocks for a double helix structure. RNA sequences are written in a 5′ to 3′ direction. The 5′ end is the part of the RNA molecule that is transcribed first, and the 3′ end is transcribed last.

In alternative embodiments, chemical linking of the two separate dsRNA strands may be achieved by any of a variety of techniques. For example chemical linking of the two separate dsRNA strands may be achieved by introducing covalent, ionic or hydrogen bonds; hydrophobic interactions, van der Waals or stacking interactions; by means of metal-ion coordination, or through use of purine analogues.

In other embodiments, the internucleoside linkages or backbones may be modified using phosphorothioates, chiral phosphorothioates, phosphorodithioates, phosphotriesters, aminoalkylphosphotriesters, methyl and other alkyl phosphonates, including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these linkage, and those backbones having inverted polarity wherein the adjacent pairs of nucleoside units are linked 3′-5′ to 5′-3′ or 2′-5′ to 5′-2′. Various salts, mixed salts and free-acid forms are also included in the modifications of the internucleoside linkage.

FIG. 2A is for illustration purpose only and shows one example of low molecular weight dsRNAs. In other embodiments, the dsRNA molecule could contain other numbers of base pairs. In one embodiment, dsRNAs of low molecular weight could contain equal to or less than 15 base pairs. In another embodiment, low molecular weight dsRNAs contain a range between 1 and 14 base pairs. In another embodiment, low molecular weight dsRNAs range between 2 and 10 base pairs. In another embodiment, low molecular weight dsRNAs range between 10 and 14 base pairs. In another embodiment, low molecular weight dsRNAs range between 2 and 5 base pairs. In another embodiment, low molecular weight dsRNAs range between 5 and 10 base pairs. In one embodiment, low molecular weight dsRNAs include dsRNAs of the same size. In other embodiments, low molecular weight dsRNAs include dsRNAs with equal to or less than 15 base pairs heterogeneous pA:pU.

The dsRNA can be synthesized by methods are discussed below, e.g., by use of an automated DNA synthesizer, such as are commercially available from, for example, Sigma-Aldrich Corporation. In one embodiment, the polyA:polyU is generated to a pre-specified size of 5 bps (low molecular weight—LMW). For comparison, the polyA:polyU may also be generated to a pre-specified size of 70 bps (high molecular weight—HMW). For example, each RNA oligonucleotide is synthesized using the t-Butyldimethylsilyl (TBDMS) protected RNA monomers on a customized RNA synthesizer. Following cleavage and a deprotection the oligonucleotide (oligo) is purified by preparative ion exchange High-Performance Liquid Chromatography (HPLC). Following purification, the oligo is desalted using an ultrafiltration process. Before annealing, each oligo is analyzed by Ion Exchange-High-Performance Liquid Chromatography (IEX-HPLC) and the oligo mass is verified with electrospray mass spectroscopy. Once the oligos are annealed, the duplex is ultrafiltered to remove residual annealing salts. If endotoxins are to be tested, the oligos are tested before and after annealing. In another embodiment, synthetic polyA:polyU of heterogenic size is endotoxin-purified and size fractionated by centrifugation through membranes of a Molecular Weight (MW) cutoff (e.g., which may be performed by Amicon). In yet another embodiment, dsRNA molecules can be synthesized by other companies, such as The Midland Certified Reagent Company, etc., and/or methods.

In alternative embodiments, many other methods to synthesize dsRNA molecules could include manual or automated reactions or in vivo in another organism. The dsRNA molecules may also be produced by partial or total organic synthesis. Any modified ribonucleotide can be introduced by in vitro enzymatic or organic synthesis. The dsRNA may be synthesized by a cellular RNA polymerase or a bacteriophage RNA polymerase (e.g., T3, T7, SP6). The use and production of an expression construct are known in the art. If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

In another embodiment, low molecular weight dsRNA may also include polyinosinic-polycytidylic acid (polyl:polyC or pI:pC) strands. In one embodiment, the polyl:polyC strands may contain 15 base paris or less. Another embodiment includes 5 base pairs of polyl:polyC. Another embodiment includes heterogeneous dsRNA strands containing polyA:polyU strands as well as polyl:polyU strands. Alternative embodiment may include other compounds. In one embodiment, the percentage of polyA:polyU may range from: 0.1% to 5%; 5% to 10%; 10% to 20%; 20% to 30%; 30% to 40%; 40% to 50%; 50% to 60%; 60% to 70%; 70% to 80%; 80% to 90%; and/or 90% to 99.9%. In another embodiment, the percentage of polyl:polyC may range from: 0.1% to 5%; 5% to 10%; 10% to 20%; 20% to 30%; 30% to 40%; 40% to 50%; 50% to 60%; 60% to 70%; 70% to 80%; 80% to 90%; and/or 90% to 99.9%.

In one embodiment, the low molecular weight dsRNAs include double stranded RNA molecules of which one strand contains poly-adenine (A) only, while the other strand contains poly-uracil (U) only. In another embodiment, the low molecular weight dsRNAs include double stranded RNA molecules of which one strand contains both A and U, and the other strand contains both U and A, in which the As from one strand are paired with Us from the other strand. In one embodiment, the low molecular weight dsRNAs include double stranded RNA molecules of which one strand contains poly-inosine (I) only, while the other strand contains poly-cytosine (C) only. In another embodiment, the low molecular weight dsRNAs include double stranded RNA molecules of which one strand contains both I and C, and the other strand contains both C and I, in which the Is from one strand are paired with Cs from the other strand.

In one embodiment, the composition of low molecular weight dsRNAs or analogues comprise a purity from about 0.1 to 100%. In another embodiment, the composition of low molecular weight dsRNAs comprises a purity from about 95 to 100%. In another embodiment, the composition of low molecular weight dsRNAs comprises a purity from about 90 to 95%. In another embodiment, the composition of low molecular weight dsRNAs comprises a purity from about 85 to 90%. In another embodiment, the composition of low molecular weight dsRNAs comprises a purity from about 80 to 85%. In another embodiment, the composition of low molecular weight dsRNAs comprises a purity from about 75 to 80%. In another embodiment, the composition of low molecular weight dsRNAs comprises a purity from about 70 to 75%. In another embodiment, the composition of low molecular weight dsRNAs comprises a purity from about 65 to 70%. In another embodiment, the composition of low molecular weight dsRNAs comprises a purity from about 60 to 65%. In another embodiment, the composition of low molecular weight dsRNAs comprises a purity from about 55 to 60%. In another embodiment, the composition of low molecular weight dsRNAs comprises a purity from about 50 to 55%. In another embodiment, the composition of low molecular weight dsRNAs comprises a purity from about 45 to 50%. In another embodiment, the composition of low molecular weight dsRNAs comprises a purity from about 40 to 45%. In another embodiment, the composition of low molecular weight dsRNAs comprises a purity from about 35 to 40%. In another embodiment, the composition of low molecular weight dsRNAs comprises a purity from about 30 to 35%. In another embodiment, the composition of low molecular weight dsRNAs comprises a purity from about 25 to 30%. In another embodiment, the composition of low molecular weight dsRNAs comprises a purity from about 20 to 25%. In another embodiment, the composition of low molecular weight dsRNAs comprises a purity from about 15 to 20%. In another embodiment, the composition of low molecular weight dsRNAs comprises a purity from about 10 to 15%. In another embodiment, the composition of low molecular weight dsRNAs comprises a purity from about 5 to 10%. In another embodiment, the composition of low molecular weight dsRNAs comprises a purity from about 0.1 to 5%. Any of the above embodiments may be used separately. Any combination of the above embodiments may be used together with one another.

FIG. 2B shows the structure of 2O′-methyl analogue of 5 base pairs polyA:polyU. 2O′-methyl analogue of 5 base pairs pA:pU is double strands of pA:pU with 2O′ methylation of the nucleic bases of the same size (5 bps). A methyl group is added to the 2′ hydroxyl group of the ribose moiety of nucleosides. 2O′-methyl analogues of dsRNAs are more resistant to enzymatic digestion and have enhanced in vivo stability. In one embodiment, 2O′-methylated versions of polyA:polyU are synthesized by the same manufacturers that synthesize the polyA:polyU molecules, and tested in parallel.

FIG. 2B is for illustration purpose only and shows one exemplary analogues of low molecular weight dsRNA molecules. Another embodiment may contain the 2O′-methyl analogue of pA:pU of other numbers of base pairs. Yet another embodiment may contain the 2O′-methyl analogue of heterogeneous pA:pU of various numbers of base pairs. In one embodiment, the low molecular weight dsRNA analogues may include 2′-O-ethyl, 2′-O-propyl, 2′-O-allyl, 2′-O-aminoalkyl or other groups. In yet another embodiment, the dsRNA could be modified in other ways. Some modifications may include, but are not limited to, 2′ modifications, modifications at other sites of the sugar or base of an oligonucleotide, introduction of non-natural bases into the oligonucleotide chain, covalent attachment to a ligand or chemical moiety, and replacement of internucleotide phosphate linkages with alternate linkages such as thiophosphates. In another embodiment, dsRNA molecules could be modified with one or more chemical groups including, without limitation, methylene blue; bifunctional groups, generally bis-(2-chloroethyl)amine; N-acetyl-N′-(p-glyoxylbenzoyl)cystamine; 4-thiouracil; and psoralen.

In yet another embodiment, the dsRNA molecules at one or both of the two single strands may be modified to prevent or inhibit the degradation activities of cellular enzymes. Techniques for inhibiting the degradation activity of cellular enzymes against nucleic acids may include, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, and phosphoramidate.

In one embodiment, the dosage of low molecular weight dsRNA or the analogues ranges from 0.1 to 1000 μg/ml. In another embodiment, the dosage of low molecular weight dsRNA or the analogues ranges from 0.1 to 10 μg/ml. In another embodiment, the dosage of low molecular weight dsRNA or the analogues ranges from 10 to 50 μg/ml. In another embodiment, the dosage of low molecular weight dsRNA or the analogues ranges from 50 to 100 μg/ml. In another embodiment, the dosage of low molecular weight dsRNA or the analogues ranges from 100 to 150 μg/ml. In another embodiment, the dosage of low molecular weight dsRNA or the analogues ranges from 150 to 200 μg/ml. In another embodiment, the dosage of low molecular weight dsRNA or the analogues ranges from 200 to 300 μg/ml. In another embodiment, the dosage of low molecular weight dsRNA or the analogues ranges from 300 to 400 μg/ml. In another embodiment, the dosage of low molecular weight dsRNA or the analogues ranges from 400 to 500 μg/ml. In another embodiment, the dosage of low molecular weight dsRNA or the analogues ranges from 500 to 600 μg/ml. In another embodiment, the dosage of low molecular weight dsRNA or the analogues ranges from 600 to 700 μg/ml. In another embodiment, the dosage of low molecular weight dsRNA or the analogues ranges from 700 to 800 μg/ml. In another embodiment, the dosage of low molecular weight dsRNA or the analogues ranges from 800 to 900 μg/ml. In another embodiment, the dosage of low molecular weight dsRNA or the analogues ranges from 900 to 1000 μg/ml.

Some of the materials and examples of methods that may be used to perform the experiments as follows:

Chemicals and Reagents: DMEM/F12, RPMI-1640 and EMEM medium are purchased from Wisent Inc. (Quebec, Canada). BEGM Bullet kit was vended by Lonza (distributed by VWR, Mississauga, Canada). RNase and DNase Free water was provided by Teknova (Hollister, Calif., USA). Fetal Bovine Serum FBS, phosphate buffered salince (PBS), 0.25 Trypsin-EDTA, dimethyl sulfoxide (DMSO), Poly (A:U), Poly (I:C), LPS and collagen type I were from Sigma-Aldrich (Steinheim, Germany). RNase Free PBS, B-27 serum-free supplement, MTT reagent, FITC AnnexinV/Dead cell apoptosis kits were from Invitrogen (Burlington, Canada). Bio-Plex Human Cytokine kits were customized by Bio Rad (Mississauga, Canada).

Cells and Cell Culture: The human hepatocellular carcinoma cell lines Huh7, HepG2 and PLC/PRC/5, human normal liver cell line THLE-2, human acute monocytic leukemia cell line THP-1, and mouse liver cancerous cell line BNL 1ME A.7R.1(ATCC cat#Tib-75) were obtained from ATCC (US). Huh7, PLC/PRC/5 and BNL 1ME A.7R.1 were grown in DMEM/F12 supplemented with 10% FBS, THP-1 in RPMI-1640 with 10% FBS. THLE-2 and human HCC xenograft cells were cultured in BEGM bullet kit, on the collagen type I coated cell culture surface. All cell cultures were kept at 37° C. in an atmosphere of 95% humidified air and 5% carbon dioxide.

Analysis of Growth Curve: Cell lines were seeded at different initial densities in the 96-well plate for 7 days. The medium was changed every day. The viability and metabolic rate of cultured cells was determined daily by assaying the reduction of 3-(4,5-dimethyl thiazol-2-yl)-2,5-diphenyl tetrazolium bromide (MTT) to formazan. After incubation period, MTT was added into each well in final concentration of 0.5 mg/ml, and incubated at the same condition for 4 h. Then the formazan was collected and dissolved in 120 ul DMSO. The absorption was measured at 560-562 nm in an ELISA reader with 620 nm as reference wave.

Analysis of Cytotoxicity and Anti-Proliferative Effect on Cells: Cytotoxicity was measured by MTT assay. Cells were seeded with the density of 5×103 cells/well in 96-well plate and incubated for 48 hours in a cell culture incubator, before being treated with compounds. Each compound was dissolved in RNase and DNase free PBS, and diluted to certain concentrations by the same PBS before being added into the cell culture supernatant. The final concentrations of 5 bps pA:pU were 50, 100, 150 and 200 μg/ml, and the gradient of 5 bps pA:pU s analogue was 50, 100, 300, 500 μg/ml. Poly(A:U) and Poly(I:C) were added at the final concentration of 200 μg/ml as controls. Cells without any treatment were the negative control. After incubation for specified times in the cell culture incubator, MTT reagent was added to the cells for the assay as described above.

Analysis of Cell Apoptosis/Necrosis: Cells were plated into 24-well plates with the density of 1×105 cells/well, and cultured for 48 hours before treatments. The 5 bp polyA:polyU and its analogue, control Poly(A:U) and Poly(I:C) were added at the final concentration of 200 μg/ml into the cell culture supernatants. Cells without treatment were the negative control and the cells treated with 1 μg/ml Lipopolysaccharide (LPS) were the positive control. After incubation for specified time, the cells were harvested and washed with ice-cold PBS, resuspended in 100 μl AnnexinV binding buffer at a concentration of 1×105 cells/100 μl and incubated with 2 μl AnnexinV-FITC for 15 minutes at room temperature in the dark. Samples were washed with binding buffer and resuspended again in 100 μl same buffer. After adding 5 μl Propidium Iodide (PI), the samples were diluted with binding buffer and analyzed by flow cytometry (BD Biosciences). Apoptotic cells were identified as an AnnexinV-FITC-positive/PI-negative population.

Analysis of Cytokines Release: Cells were plated into 96-well plate with the density of 2×104 cells/well, cultured and were allowed to become subconfluent. After changing to fresh medium, the cells were treated with 5 bp pA:pU with analogues of 5 bp pA:pU, 70 bp pA:pU, analogues 70 bp pA:pU, or control Poly(A:U) and Poly(I:C) at the final concentration of 100 μg/ml. Cells without treatment were the negative control and cells treated with 1 μg/ml LPS were the positive control. After incubation, the supernatants were collected, centrifuged and then put at −80° C. immediately. Select cytokines, such as IL-6, IL-12(p70), IFN-α2, Tumor Necrosis Factor-α (TNF-α), TNF-related apoptosis-inducing ligand (TRAIL), were analyzed by Bio-Plex Assay kits.

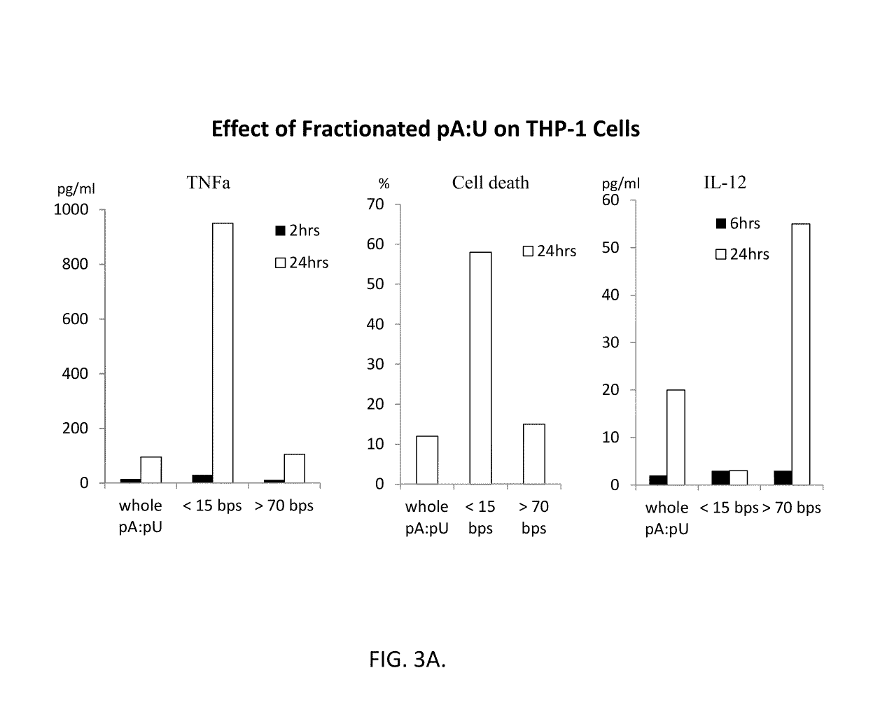

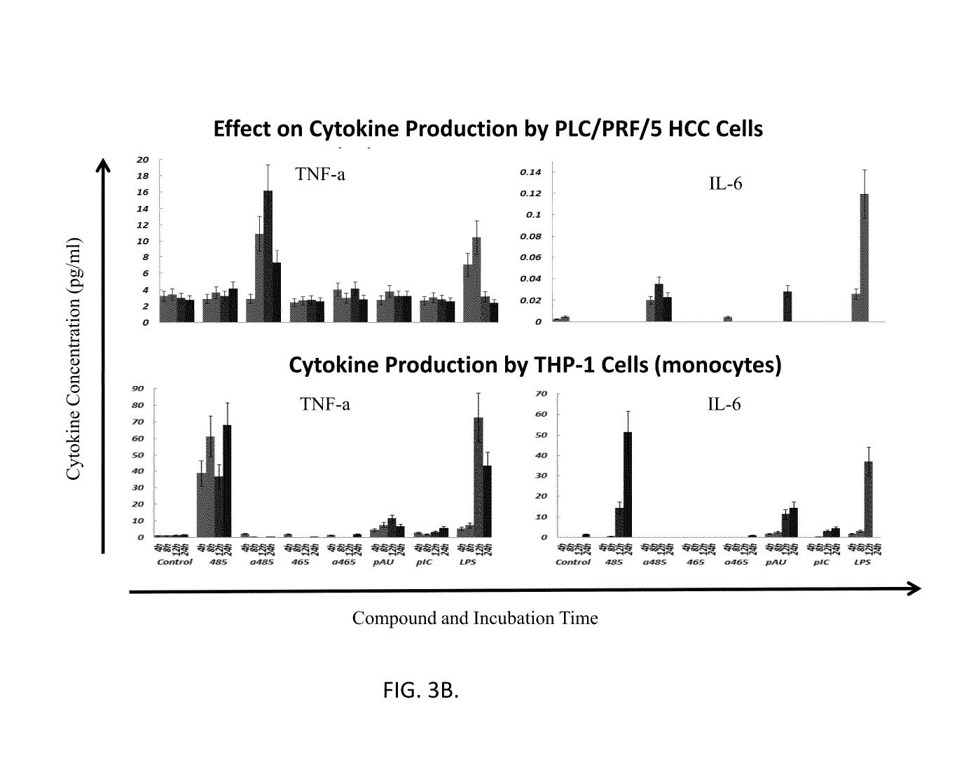

FIG. 3A shows enhanced anti-tumor cell and pro-inflammatory effects of low molecular weight dsRNA (<15 bps) on transformed cells of bone marrow origin (THP-1 cells). Experiments carried out in vitro compare low molecular weight dsRNAs (<15 bps) with high molecular weight dsRNAs (>70 bps). Data generated with dsRNA of similar size and chemical composition obtained by fractionation, indicate that low molecular weight dsRNAs (<15 bps) have substantial TNFalpha and cell death inducing properties in human monocytic THP-1 cells. In sharp contrast, size fractionated polyA:polyU of high molecular weight (>70 bps) induces high levels of IL-12p70 in human monocytic THP-1 cells, with minimal cell death or apoptosis. The pro-inflammatory effect of low molecular weight dsrnas is evaluated by measuring cytokine production using elisa (r&d systems) in FIG. 3a as well as in FIG. 3b. cell proliferation, death, and apoptosis are measured by Ethidium bromide (EB), PI and YoPro staining (in FIG. 1), Fluorescence-Activated Cell Sorting (FACS) analysis and mtt assay, Annexin V and PI staining analyzed by flow cytometry (in FIGS. 3A, 3B, 4A, 4B, 4C, 5A and 5B).

As demonstrated in FIG. 3B, low molecular weight dsRNA (5 bps pA:pU) substantially induced TNFalpha and IL-6 in human HCC cell line PLC/PRC/5 and transformed cells of bone marrow origin (THP-1 cells). Synthetic low molecular weight dsRNAs with pre-specified size of 5 bps show enhanced activity in inducing TNF-alpha and IL-6 compared with high molecular weight dsRNAs of 70 bps. 2O′-methylation of the nucleotide bases of the 5 bps pA:pU modifies the biological activity of this molecule; while the tumor cell death induction and the cytokine production by monocytes are attenuated, the induction of TNF-a by cancer cells is elevated. Synthetic dsRNAs of larger molecular weight have negligible anti-tumor cell death effect and fail to induce TNF-a and IL-6. These findings pave the way to generating novel and potent cytotoxic and pro-immunogenic agents: synthetic dsRNA of defined chemical composition and reduced molecular size.

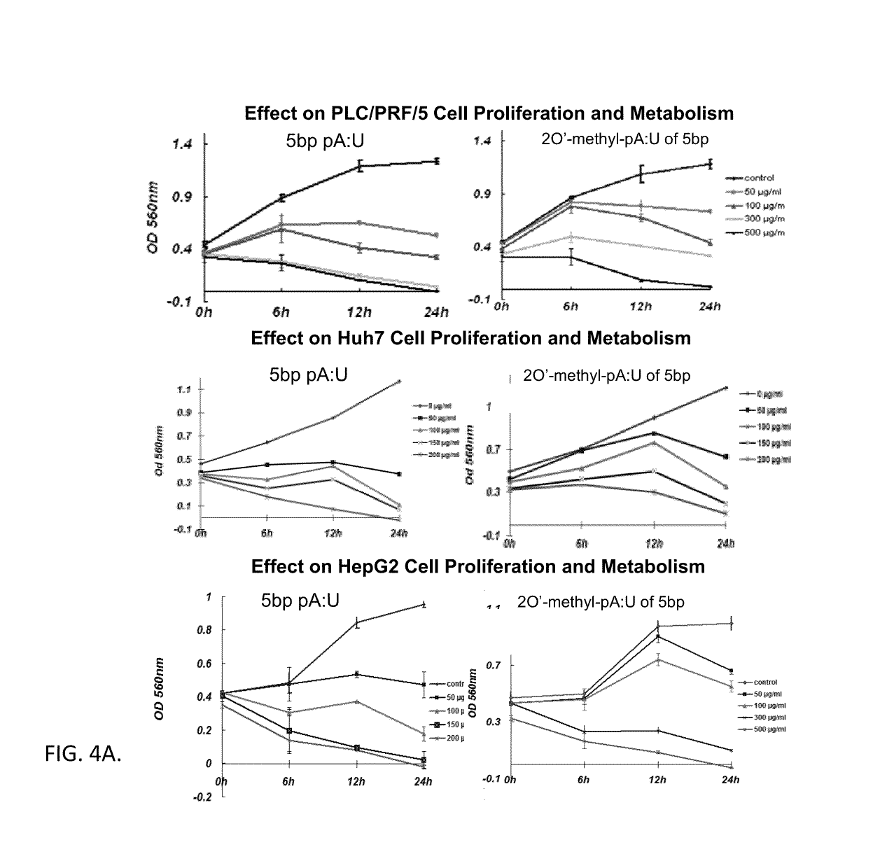

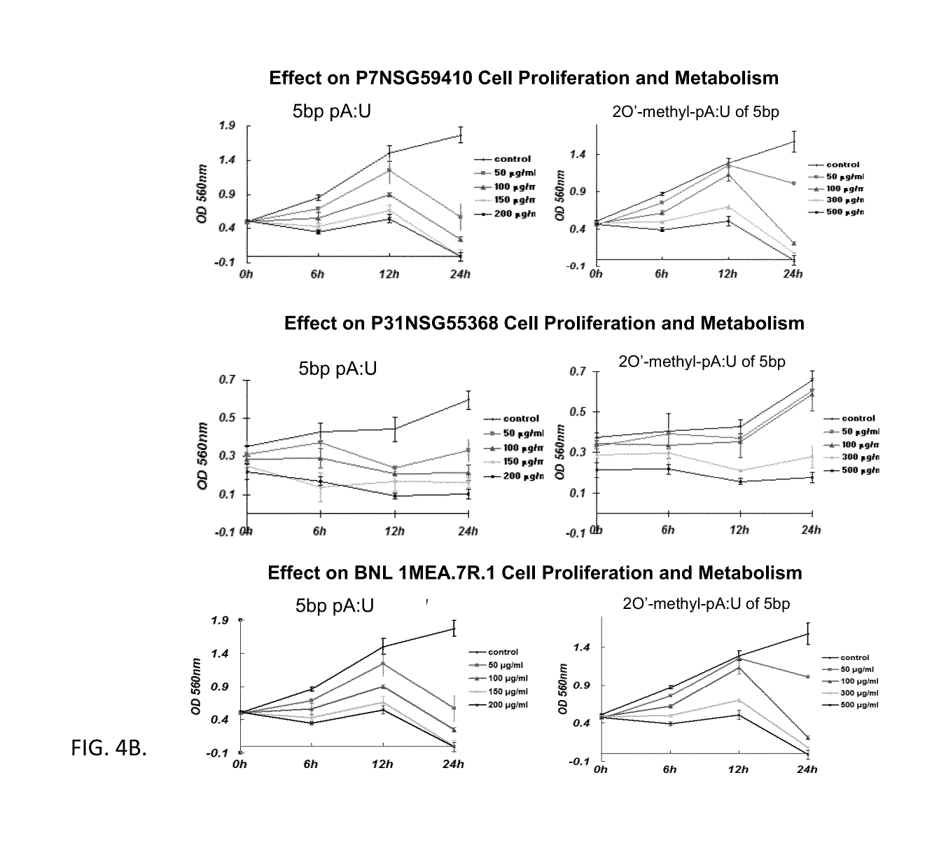

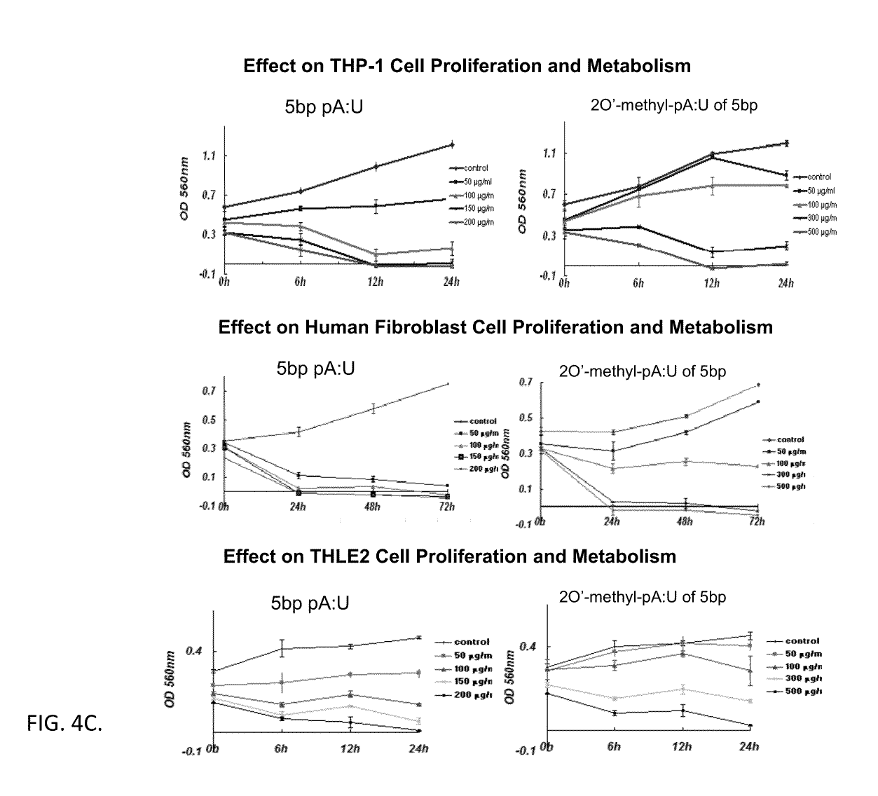

FIGS. 4A and 4B shows that polyA:polyU of 5 bps has cell growth inhibition and death inducing properties in human HCC lines Huh7, PLC/PRF/5, HepG2, primary liver cancer cells P7NSG59410 and P31NSG55368, in a dose-effect fashion. FIG. 4C shows polyA:polyU of 5 bps has cell growth inhibition and death inducing properties in THP-1 in a dose-effect fashion, while THLE2 cells emulating normal human liver cells were more refractory to 5 bp polyA:polyU; human primary fibroblasts were sensitive to 5 bp polyA:polyU. The following cell types and cell lines have been used: human liver cancer cell lines (Huh7, PLC/PRF/5, HepG2), primary human liver cancer cells (P7NSG59410 and P31NSG55368), and other cells as controls: primary human fibroblasts, mouse liver cancer cell line (BNL 1.ME A.7R.1) (ATCC cat# Tib-75). Synthetic low molecular weight polyA:polyU of 5 bps shows in vitro dose-effect cytotoxicity in three distinct human hepatocellular carcinoma cell lines, and primary liver cancer cells, and to a lesser extent in non-cancerous liver hepatocytes. The ED50 is in the 50-100 ug/ml range. These intrinsic biological properties are significantly attenuated by chemical modification consisting in 2O′-methylation of the nucleic bases, as shown in FIGS. 4A, 4B and 4C.

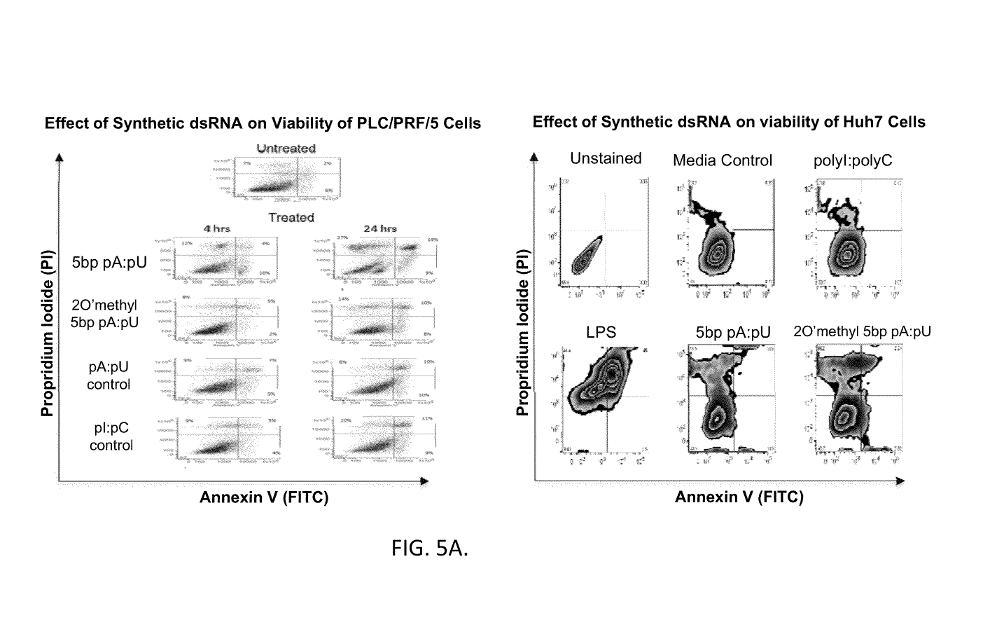

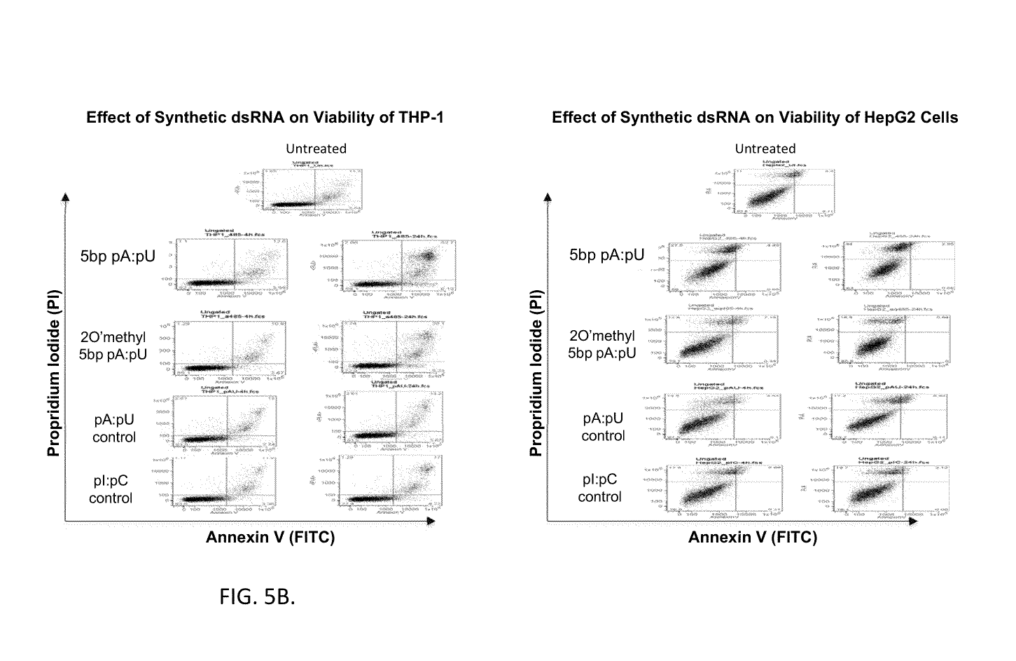

FIG. 5A shows that polyA:polyU of 5 bps induces cell death in human HCC cell line PLC/PRC/5 and human HCC cell line Huh7, while 2O′-methyl polyA:polyU analogues of 5 bps shows an attenuated cytotstatic/cytotoxic profile. In contrast, the control pA:pU, polyinosinic:polycytidylic acid (pI:pC) shows no cytotoxicity or anti-proliferative effect, induced minimal cell death or apoptosis.

FIG. 5B shows that polyA:polyU of 5 bps induces cell death in human HCC cell line HepG2 and transformed cells of bone marrow origin (THP-1 cells), while 2O′-methyl polyA:polyU analogues of 5 bps shows an attenuated cytotstatic/cytotoxic profile. In contrast, the control pA:pU, pI:pC shows no cytotoxicity or anti-proliferative effect, induced minimal cell death or apoptosis.

The primary mechanism of cytotoxicity of low molecular weight dsRNA is cytolysis, with a minimal apoptosis component. Cytotoxic evaluation on human THP-1 monocytes in FIGS. 3A and B shows an expected anti-proliferative, pro-death effect accompanied by cytokine release using low molecular weight dsRNA (5 bps). A cell line emulating normal hepatocytes has a more attenuated cytotoxicity profile and blunted TNF-alpha production upon exposure to low molecular weight dsRNA of 5 bps. In contrast, higher molecular weight dsRNA (70 bps pA:pU) compounds (native and 2O′ methyl analogue) and the unpurified control pA:pU, pI:pC have no cytotoxicity or anti-proliferative effect, and also induce minimal cell death or apoptosis with negligible TNFalpha and IL-6 inducing capabilities, as shown in FIGS. 3A, 3B, 5A and 5B. However, 70 bps pA:pU shows induction of variable levels of IFN-alpha2 in select cell lines.

The experiments indicate that 5 bps pA:pU could have a pleiotropic mechanism, distinct from that of higher molecular weight (for example, >70 bp) which is more commonly evaluated: induction of tumor cell death upon direct exposure, while normal cells are minimally affected. In this case, production of TNF-alpha by cancer cells results in amplified tumor cell death and a localized immune reaction that has the potential to generalize and curb progression of aggressive or metastatic cancer.

Low molecular weight synthetic dsRNA could have both direct tumor cytolytic and indirect immune stimulating properties. A possibility is that low molecular weight RNAs are rapidly internalized and interfere with the mRNA or miRNA management apparatus, or interact rapidly with stress sensors that control retention of pre-formed TNF-alpha through Extracellular-signal-Regulated Kinases 1/2 (ERK1/2) dependent signaling [8]. In turn, the TNF-alpha released induces rapidly growth arrest and cell death in an autocrine fashion. In addition or alternatively, other related death inducing factors such as TRAIL (TNF-related apoptosis-inducing ligand) could be rapidly mobilized, leading to activation of relevant signaling pathways and cell death [9]. The caspase dependent pathway leading to pyroptosis could also be deployed alternatively or in addition to the mechanisms outlined above.

There are two different embodiments for the tested pA:pU respectively: a native version and 2O′-methyl analogues that are more resistant to enzymatic digestion and have enhanced in vivo stability. The 2O′-methyl analogue is less potent by in vitro testing than the native compound. However, the lower pentcy of 2O′-methyl analogue in in vitro testing does not rule out that the analogue—possibly endowed with higher stability in vivo, which could have a more pronounced anti-tumor effect in a preclinical model and in vivo, in general. 5 bps pA:pU also shows a cytotoxic or anti-proliferative effect on normal fibroblasts, but only a modest effect on a cell line modeling primary human hepatocytes.

In one embodiment, various formulations have been used to deliver dsRNA or polynucleic acids, which are all in the scope of this specification. Examples of formulations for delivering dsRNA or polynucleic acids, that have been shown to be applicable to target mediated delivery, are dendrimers made of polynucleic acids, polymers in general, other biodegradable and biocompatible substances [19-32], gold based nanoparticles [21], lipid-based particles [23], lipid-based vehicles such as liposomes, silica based particles [25,26], poly(lactic-co-glycolic) acid (PLGA) based particles [28], poly(amidoamine) dendrimers [30], dendrimers constructed of other types of compounds, polyvinyl alcohol microspheres [31], and other particle formulations. Particles or vehicles for delivering dsRNA or polynucleic acids—may be of a variety of sizes varying from nm to μm—and may be coupled with antibodies, antibody fragments, aptamers, peptides and other ligands for targeting purposes. Some of the particles used to deliver the dsRNA or polynucleic acids may contain chemotherapeutic agents co-formulated with dsRNA, to achieve a more potent therapeutic effect by employing multiple mechanisms of action [19, 30-32].

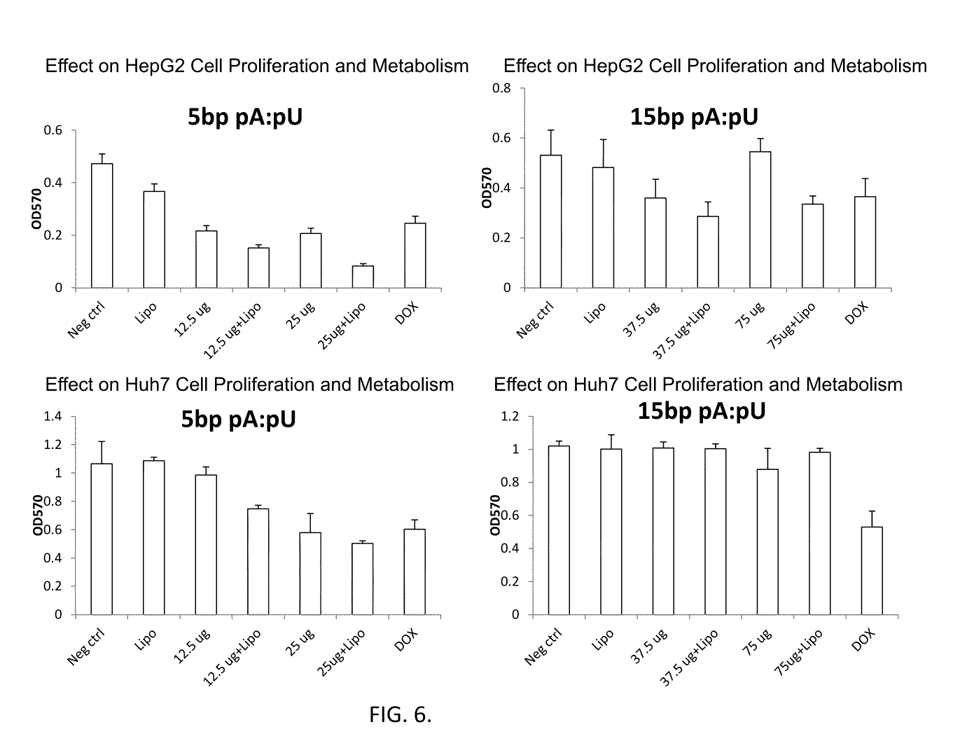

FIG. 6 shows that Lipofectamine formulated pA:pU for intracellular delivery is more biologically active than unformulated pA:pU in human liver cancer cell lines Huh7 and HepG2. Lipofectamine or Lipofectamine 2000 is a transfection reagent, produced and sold by Invitrogen. Lipofectamine may increases the transfection efficiency by lipofection. Lipofectamine reagent contains lipid subunits that can form liposomes in an aqueous environment, which entrap the transfection materials, i.e. DNA plasmids. Lipofectamine is a cationic liposome formulation that complexes with negatively charged nucleic acid molecules (to overcome the electrostatic repulsion of the cell membrane). Lipofectamine's cationic lipid molecules may be formulated with a neutral co-lipid (helper lipid).

In one embodiment, 5 bp dsRNAs are formulated using Lipofectamine 2000 to form lipid-based nanoparticles. One exemplary method follows the steps of: 1) in a microfuge tube 1.5 ul of Lipofectamine was diluted to a final volume of 23.5 ul using the appropriate media (per cell type); 2) 25 ul of the appropriate 5 bp dsRNAs analog dilution (to achieve the desired final per well concentration) was added to the diluted Lipofectamine and the mixture was incubated at Room Temperature (RT) for 5 minutes to form lipo-complexes; 3) 250 ul of the appropriate media was added to achieve a final volume of 300 ul; 4) 100 ul of the mixture from step 3 was added to each replicate and the cells was incubated for 24 hours.

Huh7 and HepG2 Cells were plated in a 96 well plate at a density of 2500 cells per well. Cells were incubated for 24 hours. After 24 hours of incubation, the medium was removed and cells were treated with various formulations of 5 bp pA:pU in triplicate in their respective culture mediums with heat activated 10% FBS. Untreated cells and Dox (10 uM) were used as controls. Cells were incubated for an additional 24 hours. After the 24 hour drug treatment, the medium was removed. Fresh culture medium will be added and the MTT assay was performed using the Life Technologies Vybrant MTT kit. After the initial reagent treatments, the Formazan was dissolved in HCl-SDS for 4 hours. The plate was read at 570 nm.

The results from MTT assay indicate that Lipofectamine formulated pA:pU for intracellular delivery is more biologically active than unformulated pA:pU. Also, it is observed that unformulated 5 bps pA:pU is more biologically active than 15 bps on both cell lines. It should be appreciated that FIG. 6 only shows an example of formulating dsRNA molecules, which should not be used to limit the scope of the invention. In other embodiments, different materials or methods could be used to obtain formulated low molecular weight dsRNAs.

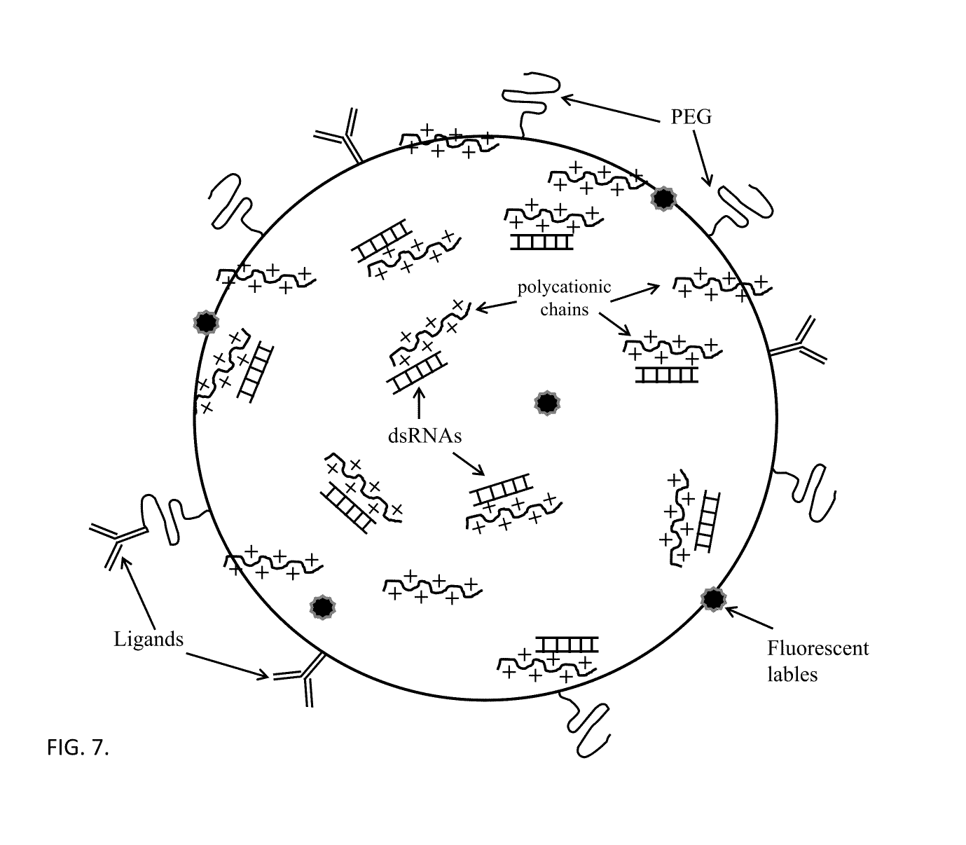

FIG. 7 shows the structure of one example of the formulated dsRNAs using biodegradable matrix. In FIG. 7, dsRNA molecules are complexed to polymer matrix through positively charged polycations. Ligands, PEG and fluorescent labels are also attached to the matrix. In other embodiments, formulated dsRNAs may not have all of the components demonstrated by FIG. 7 or may have other components instead of and/or in addition to those elements shown in FIG. 7.

In one embodiment, the dsRNAs shown in FIG. 7 include 5 bp polyA:polyU strands. In another embodiment, the formulations could include low molecular weight dsRNAs (e.g., <15 bps). In ther embodiments, the formulations could include dsRNA strands of other sizes.

In one embodiment, the dsRNAs shown in FIG. 7 include 5 bp polyA:polyU strands. In another embodiment, the formulations could include low molecular weight dsRNAs (e.g., <15 bps). In ther embodiments, the formulations could include dsRNA strands of other sizes.

In one embodiment, the compositions encompass dsRNAs formulated in particles that have a biodegradable matrix. For example, dsRNAs may be formulated using cationic polymers, lipids and polyamino acids, which forms biodegradable matrix and could protect dsRNA molecules from non-specific interactions and enzymatic degradation in the systemic circulation, and/or facilitate the delivery of dsRNAs. The cationic charge of the matrix allows electrostatic interaction with the anionic nucleic acid molecules, such as dsRNAs that leads to effective condensation. In one embodiment, dsRNAs could be attached to low- and high-molecular weight poly(ethyleneimines)(PEI), cationic poly-saccharides, chitosan, cyclodextrin, protamine, gelatin, atelocollagen, polypeptides such as poly-(L-lysine) (PLL), poly-D,L-lactide-co-glycolide (PLGA), poly(alkylcyanoacrylate), polyarginines, various cationic lipids, or dendrimers.

In one embodiment, dsRNA molecules could be formulated in dendrimers matrix. Dendrimers, which are repetitively branched molecules, may form a structure comprising a central core molecule that acts as a root, from which a number of highly branched, tree-like arms originates in a symmetrical manner. In one embodiment, dendrimers may be synthesized, via divergent methods, which include outward, repeated addition of monomers or branching, starting from a multifunctional core. Alternatively, dendrimers could be made by convergent synthesis, which includes inward branching from the dendrimer surface to the inner core by formation of individual dendrons. The dsRNA molecules could be complexed to the polycation chains, or via linkers. In alternative embodiments, dendrimers could be formulated using DNA polymers, polyamidoamine (PAMAM), modified PAMAM, polyethylene glycol (PEG), PAMAM-PEG-PAMAM, polypropylene imine (PPI) or PEI.

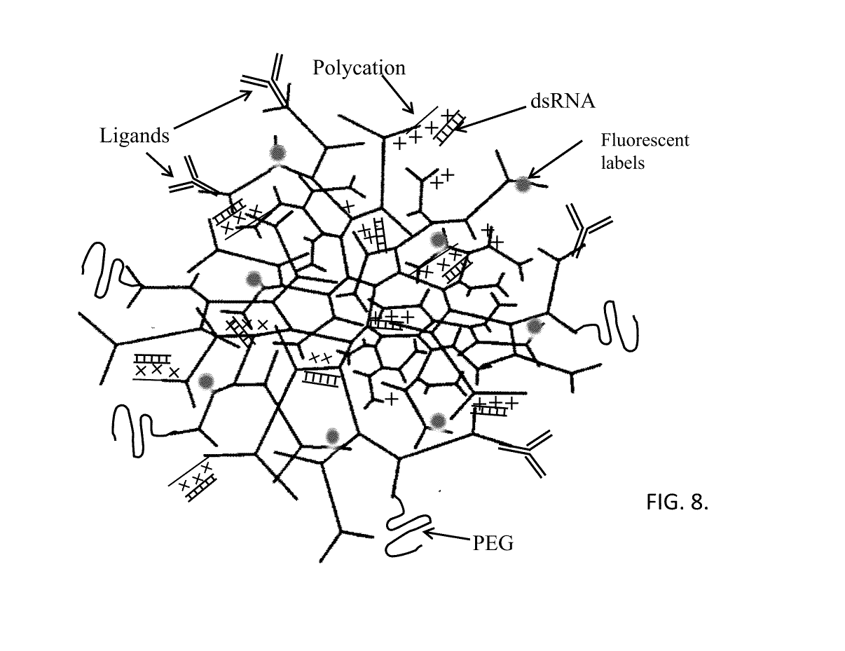

FIG. 8 shows the structure of one example of the formulated dsRNAs using dendrimers. In FIG. 8, dsRNA molecules are complexed to dendrimer matrix through positively charged polycations. Alternatively, dsRNAs could be attached to the dendrimers via other linkers or via hybridization. Ligands, PEG and fluorescent labels are also attached to the dendrimers matrix. In other embodiments, formulated dsRNAs may not have all of the components demonstrated by FIG. 8 or may have other components instead of and/or in addition to those elements shown in FIG. 8.

In one embodiment, the dsRNAs shown in FIG. 8 include 5 bp polyA:polyU strands. In another embodiment, the formulations could include low molecular weight dsRNAs (<15 bps). In other embodiments, the formulations could include dsRNA strands of other sizes.

In one embodiment, the nanoparticle formulations contain DNA dendrimers formed by joining several layers of DNA monomers. In one embodiment, the DNA monomer is formed using two single stranded DNA strands with a central region of complementary nucleotide sequence and four arms of noncomplementary nucleic acid sequence that extend from the central region. The arms of the monomer are designed to base-pair with the arms of other monomers in a precise fashion to produce several layers that interact to form a complete dendrimers. In another embodiment, the DNA dendrimers could contain one, two, three, four, or more layers of monomers.

In one embodiment, dsRNA molecules are attached to the matrix, via the use of polycationic chains or compounds via charge-charge interactions (as shown in FIGS. 7 and 8). In other embodiments, dsRNAs could be attached to the matrix, via a disulfide bridging bound; via the use of N-hydroxysuccinimide (NHS) ester dependent condensation reaction; via direct or indirect hybridization of the dsRNA to the polymers, for example, by annealing, or via other methods. Details of attaching the dsRNA to dendrimers are further described in the patent (US20120122800A1), for example.

In additional embodiments, the formulated dsRNAs could recognize specific targets to facilitate the delivery of dsRNAs. In one embodiment, the targets may include receptors, peptides, lipids, nucleic acids, metal ions, or other compounds. In alternative embodiments, the targets are selectively expressed on the tumor cells, underlying vasculature or other stromal cells. Targets associated with liver cancer vasculature such as Intercellular Adhesion Molecule 1 (ICAM-1) and Vascular adhesion protein 1 (VAP-1) have been previously described [14]. Other targets can be associated with cancer cells, and quite specific to liver cancer cells, such as glypican [15] or more general, upregulated in a variety of cancer cells, such as transferrin [16,17]. Alternatively, other targets, such as epidermal growth factor receptor (EGFR), folate, CD71, platelet endothelial cell adhesion molecule-1 (PECAM-1), frizzled family receptor 7 (FZD7), etc. could also be utilized. Still other targets could be associated with other stromal cells such as, such as Familial Adenomatous Polyposis (FAP) [18]. Also, targets could be associated with immune infiltrating cells, such as tumor associated macrophages, myeloid derived suppressor cells or dendritic cells—as these express a range of receptors capable to internalize such nanoparticles if targeted through receptors for the Fc portion of immunoglobulins (FcR), lectins, Toll-Like Receptors (TLRs), scavenger receptors, and other receptors.

In one embodiment, the formulated dsRNA particles or vehicles may contain ligands, which include antibodies, antibody fragments, aptamers, peptides, nucleotides, metal ions, heme groups or many other ligands, or any combinations hereof. In one embodiment, formulated dsRNAs can be coupled with ligands for cellular receptors. In additional embodiments, the compositions may also contain ligands for receptors preferentially expressed on tumor cells or underlying stroma, or tumor vasculature. In another embodiment, formulated dsRNAs can be generated targeting peptides or other markers that are selectively expressed on the tumor cells, underlying vasculature or other stromal cells.

In yet another embodiment, the formulated dsRNA particles or vehicles contain fluorescent agents. In another embodiment, the formulated dsRNAs could be coupled with fluorescent dye or agents, digoxigenin, fluorochromes, fluorescein or fluorescein derivatives, biotin or biotin derivatives, or other labeling molecules, compounds or groups. Alternatively, the fluorescent agent could assist tracking of the formulated dsRNAs in vitro or in vivo.

In alternative embodiments, many different materials or compounds could be attached or linked to the formulated dsRNA particles or vehicles, such as, but not limited to, a protein, a peptide, a DNA strand, a RNA strand, an aptamer, a fluorescein or fluorescein derivative, a fluorescent dye, a digoxigenin, a cholesterol, an amine, a hydrocarbon spacer, fluorescein isothiocyanate (FITC), poly-(ethylene glycol) (PEG), biotin or biotin derivative, or any combination thereof. In another embodiment, the formulated dsRNAs include protective groups, compounds, molecules and/or agents, which protects the formulations against degradation and increase the stability. Alternatively, the formulated dsRNAs are protected against degradation in body fluids, such as serum, blood plasma, etc. In another embodiment, the formulated nanoparticles are decorated with hydrophilic polymers, such as poly(ethylene glycol) (PEG), which function as shields to protect the nanoparticles from exposure to enzymes or opsonizing proteins in the systemic circulation, or help direct the particle to desired target cells. In another embodiment, the formulated dsRNAs contain a Minko group for reducing the cytotoxicity of the nanoparticles by neutralizing the positive charge of the particles. In other embodiments, matrix of nanoparticles could include inorganic nanomaterials such as gold, iron oxide nanoparticles, quantum dots or carbon nanotubes.

In one embodiment, the formulated dsRNA particles have a sizes varying from nm to μm. In another embodiment, the size of dsRNA particles are less than 100 μm. In yet another embodiment, the size of dsRNA particles are in the range of 1 μm to 100 μm. In yet another embodiment, the size of dsRNA particles are in the range of 40 nm to 1 μm. In yet another embodiment, the size of dsRNA particles are less than 40 nm. In yet another embodiment, the size of dsRNA particles are between 80 nm and 200 nm. In yet another embodiment, the dsRNA formulated particles could have other size ranges.

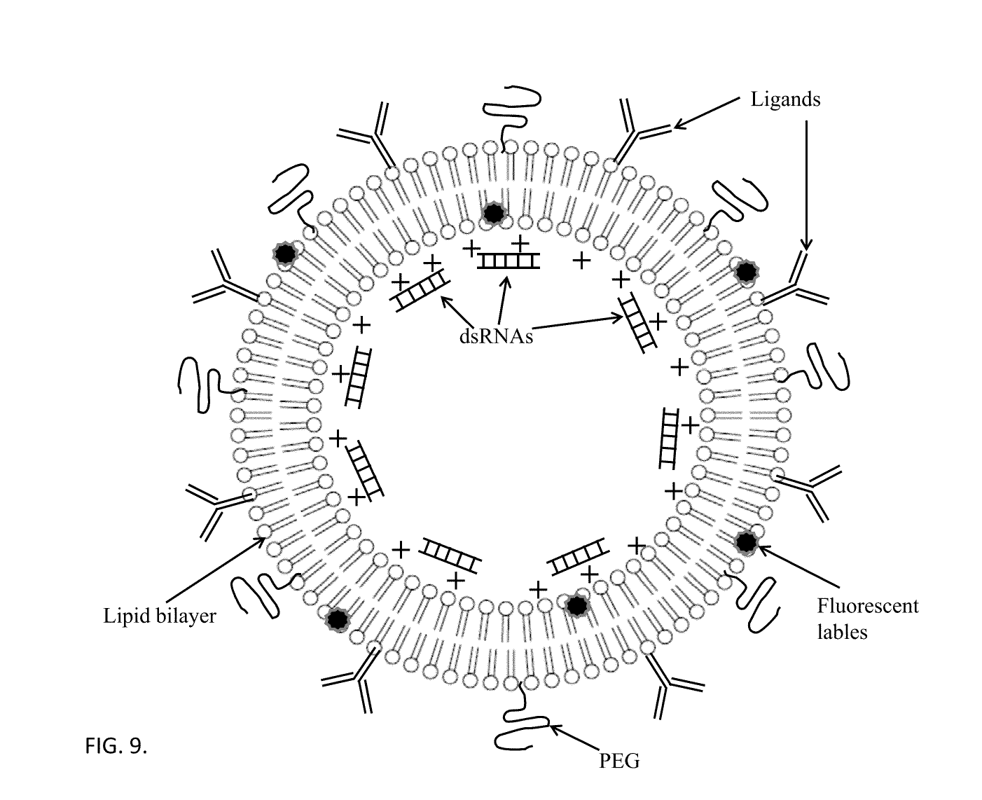

FIG. 9 shows the structure of another example of the formulated dsRNAs. In FIG. 9, dsRNA molecules are complexed with positively charged lipids encapsulated in liposomes. Ligands, PEG, and fluorescent labels are also attached to the liposomes. In other embodiments, formulated dsRNAs may not have all of the components demonstrated by FIG. 9 or may have other components instead of and/or in addition to those shown in FIG. 9.

In one embodiment, the dsRNAs shown in FIG. 9 include 5 bp polyA:polyU strands. In another embodiment, the formulations could include low molecular weight dsRNAs (e.g., <15 bps). In ther embodiments, the formulations could include dsRNA strands of other sizes.

In one embodiment, dsRNAs are formulated with lipids. In another embodiment, the formulated dsRNAs are formulated in liposomes. In yet another embodiment, the dsRNAs are formulated in immunoliposomes. In alternative embodiments, the lipids and/or liposomes include neutral (e.g., dioleoylphosphatidyl ethanolamine (DOPE), dimyristoylphosphatidyl choline (DMPC), distearolyphosphatidyl choline), negative (e.g., dimyristoylphosphatidyl glycerol (DMPG)) and/or cationic lipids or compounds (e.g., dioleoyltetramethylaminopropyl (DOTAP) and dioleoylphosphatidyl ethanolamine (DOTMA)). In alternative embodiments, dsRNAs may be encapsulated within liposomes or other vehicles and/or may form complexes thereto, in particular to cationic liposomes. In other embodiments, dsRNAs are formulated with fatty acids, fatty acid esters, steroids, chelating agents and surfactants. In alternative embodiment, the dsRNAs are formulated by transfection reagents, such as transfectamine. In yet another embodiment, dsRNAsare formulated in other ways or using other materials.

As shown in FIG. 9, dsRNAs are complexed with positively charged lipids inside liposomes. In one embodiment, the liposome could be relatively neutral. In another embodiment, the liposome could be negatively charged.

Targets that are selectively expressed on the tumor cells, underlying vasculature or other stromal cells may help deliver biologically active molecules. In one embodiment, formulated dsRNA particles or vehicles can be coupled with antibodies, antibody fragments, aptamers, peptides and other ligands for cellular receptors. In additional embodiments, the compositions may also contain ligands for receptors preferentially expressed on tumor cells or underlying stroma, or tumor vasculature. In another embodiment, formulated dsRNAs can be generated targeting peptides or other markers that are selectively expressed on the tumor cells, underlying vasculature or other stromal cells. Targets associated with liver cancer vasculature, such as ICAM-1 and VAP-1 have been previously described [14]. Other targets can be associated with cancer cells, and quite specific to liver cancer cells, such as glypican [15] or more general, upregulated in a variety of cancer cells, such as transferrin [16,17]. Alternatively, other targets such as EGFR, folate, CD71, PECAM-1, etc. could also be utilized. Still other targets could be associated with other stromal cells, such as FAP [18]. Also, targets could be associated with immune infiltrating cells, such as tumor associated macrophages, myeloid derived suppressor cells or dendritic cells—as immune infiltrating cells express a range of receptors capable to internalize such nanoparticles if targeted through FcR, lectins, TLRs, and other receptors.

In one embodiment, the liposome compositions include poly-(ethylene glycol) (PEG), which is on the surface of the liposomal carrier to extend blood-circulation time while reducing mononuclear phagocyte system uptake. Alternatively, the formulated dsRNAs are protected against degradation in body fluids, such as serum, blood plasma, etc. In alternative embodiments, the formulated dsRNAs contain a protein, a peptide, a DNA strand, a RNA strand, an aptamer, a fluorescein or fluorescein derivative, a fluorescent dye, a digoxigenin, a cholesterol, an amine, a hydrocarbon spacer, FITC, PEG, biotin or biotin derivative, or any combination thereof. In another embodiment, the formulated dsRNAs include protective groups, compounds, molecules and/or agents, which protects the formulations against degradation and increase the stability.

In yet another embodiment, some of the formulated particles may contain chemotherapeutic agents co-formulated with dsRNA, to achieve a more potent therapeutic effect by employing multiple mechanisms of action [19, 30-32].

In another embodiment, the dsRNAs could be directly conjugated with a ligand. In one embodiment, a hydrophobic ligand could be conjugated to the dsRNA to facilitate direct permeation of the cellular membrane and/or uptake across the cells. In another embodiment, the ligand conjugated to the dsRNA is a substrate for receptor-mediated endocytosis. In another embodiment, cholesterol could be conjugated to dsRNAs. Other lipophilic compounds that could be conjugated to oligonucleotides include: 1-pyrene butyric acid, 1,3-bis-O-(hexadecyl)glycerol, and menthol. Yet other ligands that may be conjugated to oligonucleotides include polyethylene glycols, carbohydrate clusters, cross-linking agents, porphyrin conjugates, delivery peptides and lipids such as cholesterol and cholesterylamine. Examples of carbohydrate clusters include Chol-p-(GalNAc)3 (N-acetyl galactosamine cholesterol) and lipophilic lithocholic oleate-(GalNAc)3 (LCO(GalNAc)3) (N-acetyl galactosamine-3′-Lithocholic-oleoyl.

In certain instances, conjugation of a cationic ligand to oligonucleotides results in improved resistance to nucleases. Alternative examples of cationic ligands are propylammonium and dimethylpropylammonium. Interestingly, antisense oligonucleotides were reported to retain their high binding affinity to mRNA when the cationic ligand was dispersed throughout the oligonucleotide.

In other embodiments, the dsRNAs formulations could be made from any of the following materials including: aliphatic polyesters such as polylactide (PLA), poly(glycolides) (PGA), poly(ε-caprolactone) (PCL); natural-based materials such as polysaccharides or peptides; hydroxyapatite (HA); metal nanoparticles, such as gold, silver or platinum; carbon nanostructures, such as fullerenes, carbon nanotubes (CNTs), carbon nanofibres (CNFs) or grapheme, or any combinations hereof. In other embodiments, the formulated particles described above could be utilized to deliver other biologically active molecules.

Another approach to deliver genetic material with impact on tumor cell viability and resulting in induction of immune response consists in utilization of viral vectors, such as oncolytic viruses [33].

Table. 1 shows some examples of the methods or compositions to formulate dsRNAs. Table. 1 is for illustration only, and should not be used to limit the scope of the invention.

TABLE l

| dsRNA Delivery Formulations | Examples of Materials or Compounds |
|---|---|
| Liposomes | Neutral (e.g. dioleoylphosphatidyl ethanolamine (DOPE), |
| | dimyristoylphosphatidyl choline (DMPC), |
| | distearolyphosphatidyl choline); |
| | Negative (e.g. dimyristoylphosphatidyl glycerol (DMPG)); |
| | Cationic lipids or compounds (e.g. |
| | dioleoyltetramethylaminopropyl (DOTAP) and |
| | dioleoylphosphatidyl ethanolamine (DOTMA)). |
| Biodegradable matrix | Cationic polymers, lipids and polyamino acids; low-and high- |
| | molecular weight poly(ethyleneimines)(PEI), cationic poly- |
| | saccharides, chitosan, cyclodextrin, protamine, gelatin, |
| | atelocollagen, polypeptides such as poly-(L-lysine) (PLL), |
| | poly-D,L-lactide-co-glycolide (PLGA), |
| | poly(alkylcyanoacrylate), polyarginines, various cationic lipids, |
| | polysaccharides, peptides, fatty acids, fatty acid esters, |
| | steroids, chelating agents and surfactants. |
| Dendrimers or micelles | Polyamidoamine (PAMAM); modified PAMAM; polyethylene |
| | glycol (PEG); PAMAM-PEG-PAMAM; polypropylene imine |
| | (PPI); polyethyleneimines(PEI); DNA polymers, et al. |
| Peptide conjugated formulations | Signal peptides (e.g. TQIENLKEKG); |
| | cell-penetrating peptides (CPPs) (e.g. Tat; Penetratin; |
| | Transportan; TP10; Oligoarginine (R8); MAP; MPG; MPGα; |
| | PTD-DRBD; CADY; Chol-R9; H3K8b; POD; DMMAn-MeI, et |
| | al) |
| Artificial DNA nanostructures | DNA nanotubes, DNA tetrahedra, DNA origami nanorobot, et |
| | al. |
| Viral vectors | Such as oncolytic vesiculoviruses; retrovirus; adeno- |
| | associated viral vectors; lentivirus; |
| Lipoprotein particles | Lipoprotein particles compose of lipoproteins such as |
| | apolipoproteins, phospholipids, cholesterol, cholesterol |
| | esters, and triglycerides. |
| Lipopeptide nanoparticles(LPNs) | LPNs use lipopeptides such as cKK-E12, cKK-A12, and |
| | cKK-O12, et al. |
| Lipophilic compounds | 1-pyrene butyric acid; 1,3-bis-O-(hexadecyl)glycerol; menthol; |
| | polyethylene glycols; carbohydrate clusters; cross-linking |
| | agents; porphyrin conjugates; delivery peptides; lipids such as |
| | cholesterol and cholesterylamine. Examples of carbohydrate |
| | clusters include Chol-p-(GalNAc)3 (N-acetyl galactosamine |
| | cholesterol) and lipophilic lithocholic oleate |
| | (LCO)(GalNAc)3 (N-acetyl galactosamine-3′-Lithocholic- |
| | oleoyl. |
| Nanosponges | Nanosponges have three-dimensional network or scaffold |
| | having backbones of long length of polyesters, embeeded in |
| | “matrix” of semibranched polyglycidol. Watersoluble |
| | polyglycidols with amino-oxy, allyl or alkyne |
| | functionalities are prepared to allow for crosslinking with |
| | polyesters, difunctionalized PEG or small molecule moieties. |
| Accurins | Accurins include a stealth and protective layer using |
| | polyethylene glycol (PEG), which is engineered to protect the |
| | Accurin from the body's immune detection and clearance |
| | mechanisms by creating a hydration shell. |
| Metal nanoparticles | Such as gold, silver or platinum nanoparticles. |
| Iron oxide nanoparticles | Magnetite (Fe3O4); the oxidized form maghemite (γ-Fe2O3), |
| | et al. |
| Quantum dots | Nanocrystal made of semiconductor materials, e.g. CdSe/ZnS |
| | QDs. |
| Carbon nanotubes | Such as fullerenes, carbon nanotubes (CNTs), carbon |
| | nanofibres (CNFs) or grapheme |
| Aliphatic polyesters | Such as polylactide (PLA), poly(glycolides) (PGA), poly(ε- |
| | caprolactone) (PCL) |
| Other types of particles or | Hydroxyapatite (HA) nanoparticle; silica based particles; |
| spheres | poly(lactic-co-glycolic) acid (PLGA) based particles; polyvinyl |
| | alcohol microspheres |

The formulation enhances, or favorably modifies the biodistribution or dual biological activity of the dsRNA within the tumor, upon systemic or topical delivery. Such compositions are desired for the treatment or management of tumors that are refractory to current therapies or relapse after standard therapy.

Some desirable features of the formulations include: (1) are safe enough to allow parenteral administration by infusion (venous, arterial) or topical administration (intra-tumoral); (2) achieve an increased bioavailability within tumor and tumor cells respectively, by virtue of having a ligand for a tumor associated receptor and (3) contain a synthetic dsRNA with exhibited one or more of immune modulating and cytotoxic modes of action when delivered through this formulation, is within the scope for this specification.

One embodiment encompasses particle formulations when the size of the particle is appropriate for intravenous, intra-arterial, or intratumoral infusion, with a desired diameter between 40 nm and 1 μM. In another embodiment, the diameter of the particle is between 80 and 200 nm. In yet another embodiment, the particles may have a size less than 100 nm. In a different embodiment, the particles have a size less than 1 um but more than 100 nm.

Some embodiments, indicate that appropriately formulated dsRNA could be superior to non-formulated dsRNA, the non-formulated dsRNA having a more diffuse biodistribution and thus expected to have a lower therapeutic index. Formulated dsRNAs would also be superior over chemotherapy alone, or formulations encompassing chemotherapies—with or devoid of specific ligands targeting cellular receptors—since chemotherapeutic agents are known to suppress rather than activate the immune system. In another embodiment, ligand engineered particles loaded with dsRNA, although similar to oncolytic viruses in respect to being cytolytic and immune activating, could be superior to the latter as they are not infectious nor have the capability to become infectious.

There is a need for therapies that achieve a superior therapeutic effect against cancer, and have an improved safety margin and therapeutic index. Low molecular weight dsRNA (e.g. equal to or less than 15 bps pA:pU) are far more potent than higher molecular weight dsRNA in regards to the direct cytotoxic effect yet the low molecular weight dsRNA retain the immune potentiating effect. Nevertheless, a formulation that has various desirable features such as: 1) increased exposure of tumor to the low molecular weight dsRNA, 2) diminished systemic exposure and 3) lack of inhibition or eventual amplification or modulation of the biological effect of dsRNAs—would be needed to focus the potent and otherwise relatively unspecific effect of the dsRNA towards the tumor and away from normal tissues.

The utilization of such low molecular weight RNAs, with strong intrinsic cytolytic capabilities, would render such formulations superior or more efficacious as compared to those described in references cited above. Such a formulation, that achieves not only tumor distribution but could introduce more effectively dsRNA into the cells, could also be applicable to higher molecular weight dsRNA and endow such molecules with a direct cytotoxic capability in addition to their intrinsic immune modulating properties.

As hepatocellular carcinoma remains an unmet medical need, current standard of care in certain clinical stages is based on trans catheter arterial chemoembolization (TACE) utilizing suspension of doxorubicin in lipiodol or drug eluting beads, with or without other approaches. While such approaches demonstrate an improvement of the clinical outlook over symptomatic treatment, novel compounds and treatments are needed to ensure a more durable management of tumor and delay or prevention of tumor relapse. Compounds with both oncolytic and immune activating properties such as low molecular weight low dsRNAs, could be superior to doxorubicin, cisplatin and other chemotherapies employed in transcatheter arterial chemoembolization (TACE), as robust activation of immunity in context of antigen release associated with cell death could have a more global and longer lasting anti-tumor effect.

The compositions described in the embodiments of the present invention are also suitable for use for the treatment of other cancers, carcinomas and malignancies. One of ordinary skill in the art, in view of the teachings of the present specification, would be able to determine dosing and modes of administration for the treatment of these conditions.

The treatment described in the embodiments could be applied to patients who express within their tumor tissue one or multiple receptors for ligands on the particles containing the dsRNA. Such ligands could be borne by co-formulated antibodies, antibody fragments, peptides or other molecules that bind to vasculature, stromal cells, cancer cells or immune infiltrating cells. Alternatively or in addition, such ligands could be borne by the matrix of the particle itself or the active molecule (dsRNA). In that case, receptors could be sensors for polynucleic acids expressed on any of the cell types mentioned above. The assessment of receptor expression within the tumor can be done with any of the standard techniques, using appropriate reagents and methodologies applied to tissue biopsies: immunohistochemistry, epifluorescent microscopy, FACS analysis, polymerase chain reaction (PCR) analysis under any of the versions of PCR (e.g. semi-quantitative Reverse Transcription-PCR (RT-PCR), or real time reverse transcription PCR (qRT-PCR), hybridization techniques and others. In all, the process, methodology and reagents described above, will be useful in identifying patients which are most likely to respond to the treatment.

In one embodiment, the low molecular weight dsRNAs, analogues or formulated dsRNA compositions may be administered topically, systematically, or by direct injection into a tumor, in solutions or in emulsions. Alternatively, examples of the administration of dsRNAs may include oral, rectal, transmucosal, transnasal, intestinal or parenteral delivery, including intramuscular, subcutaneous and intramedullary injections as well as intrathecal, direct intraventricular, intravenous, inrtaperitoneal, intranasal, intraocular or intra-cranial injection. In one embodiment, low molecular weight dsRNAs may be formulated for parenteral administration, for example by bolus injection or continuous infusion. In one embodiment, formulations for injection may be presented in unit dosage form, e.g., in ampoules or in multidose containers with optionally, an added preservative. In another embodiment, the compositions may be suspensions, solutions or emulsions in oily or aqueous vehicles, and may contain formulatory agents such as suspending, stabilizing and/or dispersing agents. In one embodiment, the dsRNAs could be dissolved in aqueous solutions in water-soluble form. In another embodiment, dsRNAs may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions. Alternatively, the active ingredients may be in powder form for constitution with a suitable vehicle, for example sterile, pyrogen-free water based solution, before use. In other embodiments, embodiments of the invention may be manufactured by processes such as, but not limited to, conventional mixing, dissolving, granulating, dragee-making, levigating, emulsifying, encapsulating, entrapping or lyophilizing processes.

Compositions of the low molecular weight dsRNAs or formulated dsRNAs may, if desired, be presented in a pack or dispenser device, such as an U.S. Food and Drug Administration (FDA) approved kit, which may contain one or more unit dosage forms. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. FDA for prescription drugs or of an approved product insert. Compositions comprising a preparation of some embodiments of the invention formulated in a compatible pharmaceutical carrier may also be prepared, placed in an appropriate container, and labeled for treatment of an indicated condition, as if further detailed above.

The amounts or dosage for administrating dsRNAs or formulated dsRNAs may range from 1 ng/kg to 999 mg/kg. Some examples of amounts or dosage may be: from 1 ng/kg to 10 ng/kg; from 10 ng/kg to 100 ng/kg; from 100 ng/kg to 500 ng/kg; from 500 ng/kg to 1 μg/kg; from 1 μg/kg to 10 μg/kg; from 10 μg/kg to 100 μg/kg; from 100 μg/kg to 200 μg/kg; from 200 μg/kg to 500 μg/kg; from 500 μg/kg to 1 mg/kg; from 1 mg/kg to 10 mg/kg; from 10 mg/kg to 100 mg/kg; from 100 mg/kg to 200 mg/kg; and/or from 200 mg/kg to 500 mg/kg.

### DESCRIPTION OF SOME EXAMPLES

One example includes low molecular weight dsRNAs (e.g., <15 bps) of heterogeneous polyA:polyU generated by size fractionation, which demonstrate substantial cytokine production inducing and cell death inducing properties. In contrast, size fractionated polyA:polyU of high molecular weight (>70 bps) induces high levels of cytokines with minimal cell death or apoptosis.

Another exempla includes synthetic low molecular weight polyA:polyU of 5 base pairs, which induces pro-inflammatory effects and cytokine production as well as substantial cell growth inhibition and cell death. In another example is 2O′-methylation of the nucleotide bases of the 5 bps pA:pU, which modifies the biological activity of this molecule.

Some other examples of the embodiments include particle formulations containing low molecular weight dsRNA, such that the particle delivers an appropriate amount of low molecular weight dsRNA to a tumor cell to induce the tumor's death in a manner associated with a cytokine inflammatory response. Particle formulations are constructed such that the particle delivers more low molecular weight dsRNA to a tumor cell as compared to low molecular weight dsRNA that is delivered as an unformulated drug absent the particle. In one embodiment, the particle formulations are made of biodegradable or biocompatible molecules such as lipids, polynucleic acids, peptides, proteins, carbohydrates, silica, metals such as gold, or other substances. Alternatively, dsRNAs may be formulated to achieve intravenous, intra-arterial, or intratumoral infusion and/or biodistribution.

In one exemplary embodiment, low molecular weight dsRNA could be formulated using lipid-based nanoparticles. In one embodiment, the lipid based nanoparticles are formed using Lipofectamine 2000.

In another embodiment, the low molecular weight dsRNAs are formulated to obtain a polymer structure. For example, low molecular weight dsRNAs would be formulated to attach to polycationic matrix to form nanoparticles. In one embodiment, the low molecular weight dsRNAs are formulated with DNA dendrimers. In another embodiment, the dsRNAs could be encapsulated in vehicles such as nanospheres. In yet another embodiment, the particles or nanospheres are made of biodegradable or biocompatible molecules.

The particles described in some embodiments of the invention contain synthetic dsRNA of defined chemical composition (polyA:polyU). In another embodiment, the particles contain synthetic 5 base pair dsRNA polyA:polyU. Alternatively, the dsRNA may contain heterogenic sizes and/or compositions. In one embodiment, the synthetic dsRNA of the payload has a defined molecular size of less than what is needed (proximately 40 bps or higher) to cross link and/or activate a Toll-like receptor. In other embodiments, the payload contains dsRNA with molecular size that is higher than the minimal size needed to cross link a Toll-like receptor. Alternatively, formulated dsRNA payload could be delivered and metabolized into strands or segments of smaller molecular weight. In yet another embodiment, the particle formulations comprise matrix that is synthetic dsRNA of defined molecular size that is higher than the minimal size needed to cross link a Toll-like receptor. In general, the payload can be synthetic dsRNA or analogue having the property of inducing cell death, or stimulating an inflammatory or immune response, or both. The particle contains a dsRNA payload that is covalently or non-covalently linked to the particle matrix. Most preferably, such particle formulations containing synthetic dsRNA are recognized by sensors such as TLR, retinoic acid-inducible gene 1 (RIG-I), Melanoma Differentiation-Associated protein 5 (MDA5) or Protein Kinase RNA-activated (PKR).

The formulation particles in one embodiment contain dsRNAs as the payload which can induce an inflammatory response consisting of TNFalpha and Interleukin 6 (IL-6). In addition, such particle formulations lead to cell death upon contact with a target cell, including but not limited to apoptosis. The particles could have a payload with other compounds or materials that leads to inhibition of proliferation of tumor cells. Alternatively, such particle formulations could be loaded with a biologically active compound or compounds that are both cell death inducing and pro-inflammatory upon formulation but are devoid of either or both effects if not formulated in the particle.

In one embodiment, formulated particles could be constructed such that the matrix of the particle includes a biodegradable substance without measurable biological effect. More specifically, the matrix of the particle could comprise a biodegradable substance without measurable biological effect itself, such as DNA without immune stimulating or immune inhibiting properties. In another embodiment, embodiments of the invention also encompass particle formulations where the matrix of the particle comprises a biodegradable substance with immune modulating properties such as unmethylated DNA containing cytosine-phosphate-guanine (CpG) palindromes.

Another exemplary embodiment encompasses a particle formulation wherein the particle encompasses a ligand for a cellular receptor expressed on cells that are part of a tumor mass, and the ligand facilitates internalization of the particle with its payload (e.g., low molecular weight dsRNA) into the tumor. In one embodiment, the ligands recognize receptors expressed on tumor vasculature, cancerous cells, stromal cells associated with the tumor, or tumor infiltrating cells of immune origin, such as tumor associated macrophages, myeloid derived suppressor cells or dendritic cells. Particles having the payload facilitate increased tumoral biodistribution and reduced systemic exposure of low molecular weight dsRNA, upon adequate infusion and compared to non-formulated low molecular weight dsRNA.

In some embodiments, the particle formulations contain a ligand linked or loaded onto the particle, in a manner that allows the ligand to facilitate the particle binding and cellular internalization in a receptor-ligand fashion. In one embodiment, the ligand can be an antibody or antibody fragment. In alternative embodiments, or in addition, the particle contains an aptamer or RNA ligand linked or loaded onto the particle, in a manner that allows the ligand to facilitate the particle binding and cellular internalization in a receptor-ligand fashion. Such ligands could be covalently linked to the particle matrix. Alternatively, such ligands could be non-covalently linked to the particle matrix. For example, some ligands include anti-ICAM-1 monoclonal antibody, anti-VAP-1 (vascular adhesion protein 1) antibody, transferin, anti-folate receptor antibody, or any ligand synthetic or natural, for receptors that could be utilized to preferentially deliver the payload to the tumor tissue or tumor cells, upon systemic or local administration.

Another embodiment includes particle formulations loaded with an antigen, such as a tumor or microbial antigen in a form of a protein. Particle formulations loaded with an antigen are capable of inducing an immune response against a tumor or microbial antigen when delivered adequately.

The particle formulation could also contain, in addition to the low molecular weight dsRNA, a chemotherapeutic agent or small molecule aimed to potentiate the therapeutic effect of the formulation.

The embodiments also encompasses a range of particles formulated with dsRNA, and with or without additional ligands, antigens and/or therapeutic agents that facilitate increased tumoral biodistribution and reduced systemic exposure of dsRNA, upon adequate infusion and compared to non-formulated dsRNA. Alternative embodiments comprise formulations with any combination of various ligands, antigens, and/or therapeutic agents. Irrespective of whether formulated particles contain added ligands, antigens and/or therapeutic agents, the particle formulations have an increased anti-tumoral activity compared to non-formulated dsRNA. Additional embodiments encompass particle formulations when the size of the particle is appropriate for intravenous, intra-arterial, or intratumoral infusion, with a desired diameter between 40 nm and 1 μM. In one embodiment, the diameter of the particle is between 80 and 200 nm. In another embodiment, the particles could have a size less than 100 nm. In a different embodiment, the particles have a size less than 1 um but more than 100 nm.

Formulated low molecular weight dsRNA are useful for the treatment of hepatocellular carcinoma. In addition, such particle formulations are useful for the treatment of tumors within the liver parenchyma, such as metastases of colon carcinoma, or other tumor types (melanoma, sarcoma, other carcinomas).

Formulated dsRNAs, with dual oncolytic and immune enhancing properties, could be positioned within the standard of care of HCC in the following way: In the therapy of patients who failed to respond to approved systemic or local therapy (TACE) with currently used agents such as doxorubicin or cisplatin.

A) As systemic targeted therapy, adequately formulated in a vehicle such as antibody targeted nanoparticle that releases the dsRNA payload preferentially at the tumor site.

B) In conjunction with TACE, as add on to currently used regimens, in non-surgically resectable tumor.

C) As an alternative to chemotherapies such as doxorubicin or cisplatin, in context of TACE.

D) As local neoadjuvant therapy to reduce tumor stage in order to render the tumor surgically resectable.

E) As adjuvant therapy (local) post resection or as an alternative to resection (in context of TACE).

Such formulations are applicable to the treatment of a wide variety of cancers that express appropriate ligands on vasculature, stromal cells, immune infiltrates or cancerous cells included in the following list.

| | | |
|---|---|---|
| Prevalent | Bladder Cancer | Lung Cancer |
| types of | Breast Cancer | Melanoma |
| cancer | Colon and Rectal Cancer | Non-Hodgkin |
| | Lymphoma | Pancreatic Cancer |
| | Endometrial Cancer | Prostate Cancer |
| | Kidney (Renal Cell) Cancer | Thyroid Cancer |

| | |
|---|---|
| | Leukemia |
| | Liver Cancer |
| Other | Acute Lymphoblastic (ALL) Leukemia |
| types of | Acute Lymphoblastic Leukemia (ALL) |
| cancers | Acute Myeloid (AML) Leukemia |
| | Acute Myeloid Leukemia (AML) |
| | Adrenocortical Carcinoma |
| | AIDS-Related Cancers |
| | AIDS-Related Lymphoma |
| | Anal Cancer |
| | Atypical Teratoid/Rhabdoid Tumor Basal Cell Carcinoma |
| | Bile Duct Cancer, Extrahepatic |
| | Bladder Cancer |
| | Bone Cancer |
| | Brain Cancer |
| | Breast Cancer |
| | Burkitt |
| | Burkitt Lymphoma |
| | Carcinoid Tumor |
| | Carcinoma of Unknown Primary |
| | Cardiac (Heart) Tumors |
| | Central Nervous System |
| | Cervical Cancer |
| | Childhood Brain Stem |
| | Chronic Lymphocytic Leukemia (CLL) |
| | Chronic Myelogenous Leukemia (CML) |
| | Chronic Myeloproliferative Disorders |
| | Colon Cancer |
| | Colorectal Cancer |
| | Cutaneous T-Cell Lymphoma |
| | Duct, Bile, Extrahepatic |
| | Ductal Carcinoma In Situ (DCIS) |
| | Endometrial Cancer |
| | Epithelial |
| | Esophageal Cancer |
| | Ewing Sarcoma |
| | Extragonadal Germ Cell Tumor |
| | Extrahepatic Bile Duct Cancer |
| | Eye Cancer |
| | Fibrous Histiocytoma of Bone, Malignant, and Osteosarcoma |
| | Gallbladder Cancer |
| | Gastric (Stomach) Cancer |
| | Gastrointestinal |
| | Gastrointestinal Carcinoid Tumor |
| | Gastrointestinal Stromal Tumors (GIST) |
| | Germ Cell Tumor |
| | Gestational Trophoblastic Tumor |
| | Giloma |
| | Hairy Cell Leukemia |
| | Head and Neck Cancer |
| | Hepatocellular (Liver) Cancer |
| | Histiocytosis, Langerhans Cell |
| | Hodgkin Lymphoma |
| | Hypopharyngeal Cancer |
| | Intraocular (Eye) Carcinoma |
| | Intraocular Melanoma |
| | Islet Cell Tumors, Pancreatic Neuroendocrine Tumors |
| | Kaposi Sarcoma |
| | Kidney Cancer |
| | Langerhans Cell Histiocytosis |
| | Laryngeal Cancer |
| | Leukemia |
| | Lip and Oral Cavity Cancer |
| | Liver Cancer (Primary) |
| | Lobular Carcinoma In Situ (LCIS) |
| | Lung Cancer |
| | Lymphoma, Primary |
| | Macroglobulinemia, Waldenström |
| | Male Breast Cancer |
| | Malignant Fibrous Histiocytoma of Bone and Osteosarcoma |
| | Melanoma, including Metastatic Melanoma |
| | Merkel Cell Carcinoma |
| | Mesothelioma, Malignant |
| | Metastatic Squamous Neck Cancer with Occult Primary |
| | Midline Tract Carcinoma Involving NUT Gene |
| | Mouth Cancer |
| | Multiple Endocrine Neoplasia Syndromes, Childhood |
| | Multiple Myeloma/Plasma Cell Neoplasm |
| | Mycosis Fungoides |
| | Myelodysplastic Syndromes |
| | Myelodysplastic/Myeloproliferative Neoplasms |
| | Myelogenous Leukemia, Chronic (CML) |
| | Myeloid Leukemia, Acute (AML) |
| | Myeloma, Multiple |
| | Myeloproliferative Disorders, Chronic |
| | Nasal Cavity and Paranasal Sinus Cancer |
| | Nasopharyngeal Cancer |
| | Neuroblastoma |
| | Non-Hodgkin Lymphoma |
| | Nonmelanoma Skin Cancer |
| | Non-Small Cell Lung Cancer |
| | Non-Small Cell Lung Cancer |
| | Oral Cavity Cancer, Lip |
| | Oropharyngeal Cancer |
| | Osteosarcoma (Bone Cancer) |
| | Osteosarcoma and Malignant Fibrous Histiocytoma |
| | Ovarian Cancer |
| | Pancreatic Cancer |
| | Pancreatic Neuroendocrine Tumors (Islet Cell Tumors) |
| | Papillomatosis, Childhood |
| | Paraganglioma |
| | Paranasal Sinus and Nasal Cavity Cancer |
| | Parathyroid Cancer |
| | Penile Cancer |
| | Pharyngeal Cancer |
| | Pheochromocytoma |
| | Pituitary Tumor |
| | Plasma Cell Neoplasm/Multiple Myeloma |
| | Primary Central Nervous System (CNS) Lymphoma |
| | Prostate Cancer |
| | Rectal Cancer |
| | Renal Cell (Kidney) Cancer |
| | Renal Pelvis and Ureter, Transitional Cell Cancer |
| | Retinoblastoma |
| | Rhabdomyosarcoma |
| | Salivary Gland Cancer |
| | Sézary Syndrome |
| | Small Cell Lung Cancer |
| | Small Intestine Cancer |
| | Soft Tissue Sarcoma |
| | Spinal Cord Cancer |
| | Squamous Cell Carcinoma |
| | Squamous Neck Cancer with Occult Primary, Metastatic |
| | Stomach (Gastric) Cancer |
| | T-Cell Lymphoma, Cutaneous |
| | Testicular |
| | Testicular Cancer |
| | Throat Cancer |
| | Thymoma and Thymic Carcinoma |
| | Thyroid Cancer |
| | Transitional Cell Cancer of the Renal Pelvis and Ureter |
| | Trophoblastic Tumor, Gestational |
| | Ureter and Renal Pelvis, Transitional Cell Cancer |
| | Urethral Cancer |
| | Uterine Cancer, Endometrial |
| | Uterine Sarcoma |
| | Vaginal CancerVulvar Cancer |
| | Waldenström MacroglobulinemiaWilms Tumor |
| | Wilms Tumor and Other Childhood Kidney Tumors |

The embodiments of the invention are further illustrated by the following examples which should not be construed as limiting in any way. The contents of all cited references including literature references, issued patents, published patent applications as cited throughout this application are hereby expressly incorporated by reference, but they are not admitted to be prior art to presently claimed invention. The practice of the embodiments of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). The above references are all incorporated herein by reference.

### EXAMPLES

Examples are provided below to further illustrate different features of the embodiments of the present invention. The examples also illustrate useful methodology for practicing the embodiments of the invention. These examples do not limit the claimed invention.

### Therapeutic Candidates and Summarized Plan

This section describes the evaluation of the in vivo pharmacological effect of formulated dsRNA in preclinical animal models encompassing tumors with an established human HCC cell line, in immunodeficient mice (subcutaneous and/or intrahepatic xenograft) and in an immune competent animal model, respectively.

TABLE 2
dsRNA candidates for in vivo evaluation:

| Molecule | Size |
|---|---|
| Double stranded polyA:polyU | 5 base pairs |
| Double stranded 2O'-methyl polyA:polyU | 5 base pairs |

TABLE 3
Research plan description:

| Aim | Steps | Outcome |
|---|---|---|
| Generation and evaluation of | Particles are compared with | Targeted |
| two or several pA:pUs in | unformulated pA:pU in terms of | nanoformulations to be |
| targeted nanosphere | biological effect, on established | tested in vivo. |
| formulations, to select | cell lines. | |
| appropriate ones to test in | | |
| vivo. | | |
| Evaluation of preclinical | Model and methodology set-up, | Preclinical pharmacology |
| activity of formulated vs | subcutaneous/orthotopic grafting | data-set. Preliminary |
| unformulated pA:pU in | of the select HCC cell lines in | evidence of safety. |
| xenograft models with | immune deficient mice or an | |
| established HCC cell lines. | alternate adequate model (cell | |
| | lines Huh7, HepG2 and | |
| | PLC/PRC/5). | |
| | 1. Test pA:pU utilizing an | |
| | intensive administration | |
| | regimen, in several | |
| | xenografts. Select one to | |
| | proceed, based on data. | |
| | 2. Optimize dosing and | |
| | compare local versus | |
| | systemic administration of | |
| | pA:pU in select xenograft. | |
| | Evaluate gross toxicities. | |
| | Select dosing protocol to | |
| | proceed with. | |
| | 3. Compare pA:pU, pA:pU | |
| | analogue and controls in | |
| | the select model with a | |
| | given dosing. Select cmpd | |
| | or analogue for further | |
| | evaluation. | |
| | 4. Identify and test a range of | |
| | targeted, slow release or | |
| | depot formulation against | |
| | non-formulated cmpd in | |
| | select xenograft. | |
| | Monitor tumor growth, morbidity | |
| | and mortality. Tumor models are | |
| | subcutaneous to allow dose | |
| | optimization and selection of cmpd | |
| | and cell line. Then validate the | |
| | final data set in orthotopic model | |
| | as applicable. | |
| Evaluate preclinical activity | Model and methodology set-up, | Preclinical immune- |
| of formulated vs | subcutaneous grafting of the | pharmacology data-set in |
| unformulated pA:pU in | select HCC cell line in immune | an immune competent |
| immune competent tumor | competent BALB/c mice (BNL | model, showing direct |
| model. | 1ME A.7R.1). | anti-tumor response and |
| Utilize a mouse cell line that | 1. Optimize dosing of | indirect, longer term |
| shows susceptibility to | formulated pA:pU to show | immunological |
| pA:pU by previous testing in | anti-tumor effect in the | protection. |
| vitro (BNL 1ME A.7R.1). | select model. Select | |
| | dosing protocol to proceed | |
| | with. | |
| | 2. Compare pA:pU, pA:pU | |
| | analogue in the select | |
| | model with a given dosing. | |
| | Select formulation with | |
| | cmpd or analogue for | |
| | further evaluation. | |
| | 3. Evaluate immunological | |
| | effect by re-challenging | |
| | the treated mice with BNL | |
| | 1ME A.7R.1 or suitable | |
| | cell line. | |
| | Monitor tumor growth, morbidity | |
| | and mortality. | |

To perform an adequate quality control of the formulated or non-formulated investigational agents prior to animal dosing, and qualify and interpret the preclinical results, the effect of formulated and unformulated dsRNAs is evaluated on the THP-1 cell line for cytotoxicity and cytokine production and as necessary, on other cells such as the tested HCC cell line used for xenograft and murine splenocytes.

### Example 1

### Generation and Characterization of Two or Several dsRNA Targeted Nanosphere Formulations, to Select an Appropriate One to Test In Vivo

Hypothesis: formulation of low molecular weight dsRNAs (5 bps pA:pU) in targeted nanosphere that previously showed enhanced miRNA delivery into target expressing cells in vitro, will enhance the biological activity of dsRNA.

Design: Particles encompassing low molecular weight dsRNA, a biodegradable matrix such as polymer (DNA) based and ligand (such as anti-ICAM-1 antibody) are generated and compared with unformulated pA:pU in terms of biological effect, on established cell lines.

Methodology: HCC cell lines, Human umbilical endothelial cells HUVEC (endothelial) cell line, and other control cell lines are exposed in a dose ranging fashion to formulated and unformulated dsRNA. Outcome in the form of impact on viability, proliferation, and cytokine production is measured using previously established methods. This is correlated with target expression profile of tested cells.

Results: The nanoparticle formulated low molecular dsRNA show increased induction of cell death and cytokine production as compared to non-formulated dsRNA. The nanoparticle formulated control high molecular dsRNA render this species of molecule cytotoxic while amplifying its immunologic properties manifested through cytokine production.

### Example 2

### Evaluation of the Preclinical Activity of dsRNA in Subcutaneous Human Xenograft Model; Validation of the Results in Orthotopic Human HCC Xenograft Model, in Immunodeficient Mice

Hypothesis: Upon local or systemic administration, formulated 5 bps pA:pU suppresses the growth of xenografts or induces tumor regression at doses that have tolerable toxicities. Such dsRNA formulated in targeted nanoparticles has the capability to enhance local biodistribution, bioactivity and safety margin of said dsRNA.

Design: Generation of preclinical proof of concept in subcutaneous and orthotopic xenografts with an established HCC cell line in immunodeficient mice. The following steps are performed:

1) Test 5 bps pA:pU (formulated vs. unformulated) utilizing iv infusion and determine maximum tolerated dose vs. efficacious dose.

2) Test 5 bps pA:pU utilizing an intensive local administration regimen, in several xenografts: (Huh7, HepG2 and PLC/PRC/5). Select one to proceed, based on data.

3) Optimize dosing and compare local versus systemic administration of 5 bps pA:pU in select xenograft. Evaluate gross toxicities. Select dosing protocol to proceed with in the next steps.

4) Compare 5 bps pA:pU, analogue and controls in the select model with a given dosing. Select cmpd or analogue for further evaluation.

5) Identify and test slow release or depot formulation versus non-formulated, and nanoparticle formulated cmpd in select xenograft, to define as needed back-up formulation approaches. Validate as applicable, the data set in an orthotopic version of the xenograft model.

Methodology: First, the xenograft is established by injection of human HCC cells or implantation of tumor tissue fragments from other mice, subcutaneously or orthotopically. Profoundly immune deficient mice are being used, as well as HCC lines for which there is already demonstrated anti-tumor cell effect by MTT or similar assays in vitro, and preferably, tumor take in any immunodeficient model. Upon tumor reaching a measurable size (for the subcutaneous implant) or demonstrable laboratory correlate for liver function impairment (for the orthotopic implant), the animals are randomized to several treatments (n>5/group) such as:
Systemic infusion of formulated vs. unformulated 5 bps pA:pU: A dose escalation approach will be employed, to define maximum tolerated dose in an acute setting. The MTD dose will be then used to evaluate the efficacy upon chronic dosing.
As a control and fall back strategy, intra-tumoral administration of 5 bps pA:pU or analogue (3×/week for up to 2 weeks unless untreated mice reach a tumor size that warrants euthanasia). A dose ranging approach is being pursued: 30 ug, 3 ug and 0.3 ug/injection.
As control, dose matched intra-tumoral administration of 70 bps pA:pU or unpurified total pA:pU, respectively (3×/week for up to 2 weeks unless untreated mice reach a tumor size that warrants euthanasia). A dose ranging approach will be pursued: 30 ug, 3 ug and 0.3 ug/injection.
Sterile saline control, intra-tumoral, utilizing the protocol/timing from above.
An appropriate positive control (chemotherapeutic agent such as doxorubicin or small molecule TKI preferably sorafenib or sunitinib) administered as necessary.

Tumor progression is monitored by caliper or appropriate measurement (lab analytes); in addition, potential dose-related toxicities are assessed by periodic evaluation of the clinical status of the animals. Following sacrifice of the animals, tumors are evaluated histopathologically, immunohistochemically, and/or by flow cytometry.

For systemic treatment, a preliminary single dose evaluation is employed to determine acute toxicity, by iv infusion of formulated vs. unformulated 5 bps pA:pU, starting with 10 ug in semilogarithmic dose escalation increments (30 ug, 100 ug, 300 ug, 1 mg) in cohorts of 5 mice per group, in the immune deficient model intended for evaluation. Upon defining the maximum tolerated dose, a preclinical evaluation is done as depicted above, with the appropriate differences regarding the dosing strategy (i.v. infusion and dose at MTD). In addition, chronic dose toxicity evaluation is performed within the target dose range. To properly qualify and interpret the data, splenocytes are tested for cytokine production (such as TNFalpha) upon incubation with various concentrations of 5 bps pA:pU.

For topical administration, once a dosing approach for non-formulated 5 bps pA:pU, associated with anti-tumor effect, has been established, nanoformulations and a depot formulation will be tested. The following candidate formulations are considered along with the nanoformulations, as they have clinical relevance: suspension of 5 bps pA:pU in lipiodol (Laboratoire Guerbet), 5 bps pA:pU adsorbed onto biodegradable beads similar to those currently used for TACE with doxorubicin (Biocompatibles PLC, Farnham, UK) or absorbable gelatin sponge (Gelfoam; Pharmacia & Upjohn, Peapack, N.J., USA)—as all these formulations are routinely used for local management of HCC and carry the promise of increasing local biodistribution of 5 bps pA:pU in conjunction with TAE (trans catheter arterial embolism).

As applicable, the data set obtained in a subcutaneous xenograft model is validated in an orthotopic model.

Toxicity assessment: For systemic treatment, a preliminary single dose evaluation is done for acute toxicity, by iv and intra-tumoral infusion of 5 bps pA:pU, starting with 10 ug in semilogarithmic dose escalation increments (30 ug, 100 ug, 300 ug, 1 mg) in cohorts of 5 mice per group, in the model intended for evaluation. Upon defining the maximum tolerated dose, a preclinical evaluation is done as depicted above, with the appropriate differences regarding the dosing strategy (i.v. infusion and dose at MTD). In addition, chronic dose toxicity evaluation is performed within the target dose range.

Results: The nanoformulated 5 bps pA:pU have an enhanced “cytoreductive” effect (tumor regression and partial or complete remission) or “cytostatic” effect (slow down or curbing tumor progression), that compares positively from a statistical standpoint with appropriate controls including non-formulated 5 bps pA:pU. This is applicable to both topical and systemic administration, and is accompanied by increased pro-inflammatory cytokine production within the tumors in animals treated with nanoformulated dsRNA.

### Example 3

### Evaluation of Preclinical Activity of 5 Bps pA:pU in an Immune Competent Tumor Model

Hypothesis: An immune deficient model could underestimate the efficacy of 5 bps pA:pU against tumors. Upon local or systemic administration, 5 bps pA:pU could suppress tumor growth or induce tumor regression of primary or secondary (remote or metastatic tumors) without dose-limiting toxicities, in immune competent mice. Proof of anti-tumor activity in immune deficient animals is complemented by additional info in a fully immune competent model.

Design: subcutaneous grafting of the select HCC cell line in immune competent BALB/c mice (BNL 1ME A.7R.1) followed by testing of short term local and longer term anti-tumoral effects against remote lesions. The following steps are performed:

1) Optimize dosing of 5 bps pA:pU to show anti-tumor effect in the select model. Select dosing protocol to proceed with in the next steps.

2) Compare 5 bps pA:pU, analogue and controls in the select model with a given dosing. Select cmpd or analogue for further evaluation.

3) Evaluate immunological effect by re-challenging the treated mice with BNL 1ME A.7R.1 at a remote site.

Methodology: This model encompasses syngeneic mouse cells (BNL 1ME A.7R.1) inoculated subcutaneously or into the hepatic tissue of BALB/c mice. The experimental design and dosing approach are similar to that described for immune deficient mice, with several exceptions.
As applicable, the local administration is performed by subcutaneous, intra-splenic, intra-hepatic or intra-peritoneal administration as feasible from a technical standpoint. This will be compared to systemic dosing.
The dosing is initiated at an interval after tumor implantation, corresponding to established lesions detectable by pathology evaluation, unless the subcutaneous route is utilized (in that case dosing is started when tumors are evaluable).
The efficacy is assessed by monitoring the status of the animals: for orthotopic tumors, analytes related to the liver function, presence of ascites or by utilizing specific imaging techniques as appropriate. When the control (untreated) group shows specific signs of disease (liver failure or terminal disease) all animals are sacrificed and analyzed from histopathological standpoint.
An experimental group is also considered in which a secondary tumor is induced and monitored for potential regression upon treatment of the primary tumor by intra-tumoral administration of 5 bps pA:pU The technical feasibility of this approach is explored. Different timing of induction of secondary tumor (delayed induction relative to induction of primary tumor and treatment), is also explored, to assess the capability of the nanoformulated pA:pU to induce immunogenic death and immune memory.

Results: Nanoformulated low molecular weight dsRNA administered systemically or directly delivered to the tumor environment has strong local direct and immune modulatory effect manifested in tumor inhibition, superior over non-formulated low molecular weight dsRNA or controls. A positive outcome is disease control as reflected by suppression of tumor progression. In addition, nano-formulated low molecular weight dsRNA has effects on remote tumors, or secondary tumors, through mobilizing the systemic immunity.

### Other Extensions or Alternatives

Each embodiment disclosed herein may be used or otherwise combined with any of the other embodiments disclosed. Any element of any embodiment may be used in any embodiment.

Although the invention has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention.

We claim:

1. A composition comprising double stranded ribonucleic acid (dsRNA) molecules, which induce tumor cell death or suppress tumor growth, wherein the double stranded RNA molecules contain equal to or less than 15 base pairs.

2. The composition of claim 1, wherein the dsRNA molecules have polyadenylic-polyuridylic acid (polyA:polyU or pA:pU) strands.

3. The composition of claim 1, wherein the dsRNA molecules have polyinosinic-polycytidylic acid (polyl:polyC or pI:pC) strands.

4. The composition of claim 1, wherein the double stranded RNA molecules has only 5 base pairs.

5. The composition of claim 1, wherein the double stranded RNA molecules has only 2 to 10 base pairs.

6. The composition of claim 1, wherein the dsRNA molecules are formulated in a formulation to facilitate the delivery of dsRNA to a tumor cell to induce death of the tumor cell or suppress growth of the tumor cell.

7. The composition of claim 6, wherein the formulation includes a matrix and the dsRNAs attach covalently or non-covalently to the matrix.

8. The composition of claim 6, wherein the formulation includes at least one of polymer-based nanoparticles, lipid-based nanoparticles, liposomes, dendrimers, micelles, peptide conjugated formulations, artificial DNA nanostructures, viral vectors, lipoprotein particles, lipopeptide nanoparticles, lipophilic compounds, metal nanoparticles, silica based particles, poly(lactic-co-glycolic) acid (PLGA) based particles, polyvinyl alcohol microspheres, nanosponges, accurins, iron oxide nanoparticles, quantum dots, carbon nanotubes, hydroxyapatite (HA) nanoparticle, or aliphatic polyesters.

9. The composition of claim 6, wherein the formulation includes a chemotherapeutic agent.

10. The composition of claim 6, wherein the formulation further comprises a ligand that binds to a peptides, lipids, or molecules or other markers that facilitates delivery of the dsRNA molecules to the tumor cell.

11. The composition of claim 10, wherein the ligand binds to a cellular receptor, which is expressed on at least one of a cancerous cell, a stroma cell, an endothelial cell, a macrophage, a myeloid derived suppressor cell, or a dendritic cell.

12. The composition of claim 1, including an amount of the dsRNA that is effective for inducing tumor cell death or suppressing tumor growth.

13. A composition comprising dsRNA molecules, which induce tumor cell death or suppress tumor growth while also inducing an inflammatory cytokine reaction, wherein the double stranded RNA molecules contain equal to or less than 15 base pairs.

14. A method comprising delivering double stranded ribonucleic acid (dsRNA) molecules, therein inducing tumor cell death or suppressing the tumor growth or inducing an inflammatory cytokine reaction, wherein the double stranded RNA molecules contain equal to or less than 15 base pairs.

15. The method of claim 14, further comprising delivering formulated dsRNA to the tumor.

16. The method of claim 14, wherein the dsRNA molecules are formulated using at least one of the methods of polymer-based nanoparticles, lipid-based nanoparticles, liposomes, dendrimers, micelles, peptide conjugated formulations, artificial DNA nanostructures, viral vectors, lipoprotein particles, lipopeptide nanoparticles, lipophilic compounds, metal nanoparticles, silica based particles; poly(lactic-co-glycolic) acid (PLGA) based particles; polyvinyl alcohol microspheres, nanosponges, accurins, iron oxide nanoparticles, quantum dots, carbon nanotubes, hydroxyapatite (HA) nanoparticle, or aliphatic polyesters.

17. The method of claim 16, wherein the formulation further comprises a ligand.

18. The method of claim 17, wherein the ligand binds to a cellular receptor, which is expressed on at least one of a cancerous cell, a stroma cell, an endothelial cell, a macrophage, a myeloid derived suppressor cell, or a dendritic cell.

19. The method of claim 14, further comprising attaching ligands to the dsRNA molecules or to a formulation of the dsRNA molecules, and treating a patient with a tumor that has receptors for the ligands.

20. The method of claim 14, including using an amount of the dsRNA that is effective for inducing tumor cell death or suppressing tumor growth.
